SEQUENCE LISTING (3 sequences)

SEQ ID NO: 1 - protein, 9 aa, Artificial Sequence
  Synthetic annexin V atherosclerotic plaque binding peptide
YRALVDTLK

SEQ ID NO: 2 - protein, 16 aa, Artificial Sequence
  Synthetic annexin V atherosclerotic plaque binding peptide
YAKFRETLEDTRDRMY

SEQ ID NO: 3 - protein, 17 aa, Artificial Sequence
  Synthetic annexin V atherosclerotic plaque binding peptide
RALVDTEFKVKQEAGAK

(19) United States
(12) Patent Application Publication
(10) Pub. No.: US 2009/0004118 A1
(43) Pub. Date: Jan. 1, 2009

(54) Multifunctional Nanoparticle Conjugates And Their Use

(76) Inventors: Shuming Nie, Atlanta, GA (US); Yong Kyu Lee, Decatur, GA (US); Gloria Kim, Atlanta, GA (US)

Correspondence Address: KLARQUIST SPARKMAN, LLP, 121 SW SALMON STREET, SUITE 1600, PORTLAND, OR, 97204, US

(21) Appl. No.: 11/664,886

(22) PCT Filed: Oct. 7, 2005
(86) PCT No.: PCT/US05/36222

§ 371 Date: Apr. 6, 2007

Related U.S. Application Data
(60) Provisional application No. 60/617,158, filed on Oct. 7, 2004.

(51) Int. Cl. A61K 49/10; C07H 5/06; C07K 16/00; A61P 35/00; C12N 5/06; C07K 2/00; A61K 31/727

(52) U.S. Cl. 424/9.35; 536/21; 530/391.9; 514/56; 435/375; 977/906

(57) ABSTRACT

Disclosed herein are conjugates comprising a nanocarrier, a therapeutic agent or imaging agent and a targeting agent. Also disclosed herein are compositions comprising such conjugates and methods for using the conjugates to deliver therapeutic and/or imaging agents to cells. Also disclosed are methods for using the conjugates to treat particular disorders, such as proliferative disorders.

### CROSS REFERENCE TO RELATED APPLICATION

This application claims the benefit of U.S. Provisional Application No. 60/617,158, filed Oct. 7, 2004, which is incorporated herein by reference in its entirety.

### FIELD

The present disclosure concerns drug delivery of therapeutic or imaging agents to a target tissue. In general the disclosed compounds include a targeting component, a therapeutic or imaging component and a nanocarrier component. The disclosure also concerns compositions containing such compounds and methods for using such compounds and compositions.

### BACKGROUND

Considerable research has been directed to discovering systems whereby a pharmaceutical agent could be selectively delivered to a desired anatomic location; namely the site in need of treatment. In spite of some progress in this area many pharmaceutical treatments of various diseases or health risks impart substantial risk to the patient due to lack of selective drug delivery. The risks are particularly acute in cancer therapy because pharmacologically active anticancer drugs reach tumor tissue with poor specificity and dose-limiting toxicity.

With respect to the treatment of cancer, drugs which are effective in attacking malignant cells to destroy them, or at least limit their proliferation, generally attack benign cells as well. Although it is desirable to concentrate a cytotoxic agent at a targeted site, current cancer treatment protocols instead involve non-specific or systemic dosing, with careful monitoring of the patient. The dose is selected to be just below the amount that will produce acute (and sometimes chronic) toxicity that can lead to life-threatening cardiomyopathy, myelotoxicity, hepatic toxicity, or renal toxicity. Alopecia (hair loss), mucositis, stomatitis, and nausea are other common, but generally not life-threatening, side effects at these doses.

Previous attempts to administer cytotoxic drugs by direct injection into the location of the organ having the malignancy are only partially effective, because of dispersion of the drug from that location. Such dispersion cannot be totally prevented, with the result that excessive quantities of drug need to be administered to attain a desired result. Although careful clinical monitoring may minimize extensive damage or loss of viable tissue, a compound that is actively transported through standard biological systems to the treatment site prior to activation of the cytotoxic agent would be highly desirable. Thus, there exists a need for a drug delivery system that can accomplish site-specific release of a therapeutic agent in target cells, tissues, or organs.

### SUMMARY OF THE DISCLOSURE

Disclosed herein are compounds including a nanocarrier, a therapeutic agent or imaging agent, and a targeting agent. The nanocarrier can be a nanoparticle, an organic polymer, or both. In one embodiment, tripartite compounds can be represented by one of the formulas

A-X—Y

X-A-Y

or

X—Y-A

wherein A represents a chemotherapeutic agent or an imaging agent;

X represents a nanocarrier that is a nanoparticle, an organic polymer or both; and

Y represents a targeting agent.

The disclosed compounds are designed to target particular cells or tissues, so that a therapeutic agent or an imaging agent can be delivered to the desired location more effectively. For example, one embodiment of the disclosure includes compounds that target cancerous tissues. As such, certain examples of these compounds include a targeting agent Y that binds to a receptor that is present in a higher concentration on cancer cells. For example, certain types of cancer cells express receptors for folate, biotin or vitamin B₁₂ at higher concentrations than normal cells. Embodiments of the disclosed compounds can exploit up-regulated receptors to deliver a therapeutic agent to a cancer cell selectively.

Certain embodiments of the disclosed compounds exploit another feature of cancerous tissues to provide selective delivery of therapeutic agents or imaging agents to such tissues. For example, embodiments of the compounds that include a nanoparticle or self-assemble to form a nanoparticle can benefit from the enhanced permeability and retention effect (EPR effect) and accumulate in tumors.

Certain organic polymers may induce the compounds to self assemble to form a self-assembled nanoparticle, which provides an effectively multivalent species. In such embodiments the self-assembled nanoparticles can include the same or different compounds. For example, the self-assembled nanoparticles can include compounds having different targeting agents, imaging agents, therapeutic agents and nanocarrier components.

Embodiments of the disclosed compounds also include plural therapeutic agents, imaging agents and or targeting agents. In such embodiments, the compounds can include different therapeutic agents, imaging agents and targeting agents. In certain embodiments compounds having plural targeting agents have increased affinity for their target due to multivalent effects.

In one embodiment, the disclosed compounds are formulated into pharmaceutical compositions for administration to a subject. For example, one aspect of the disclosure concerns treating a subject having a proliferative disorder using the disclosed compounds, and hence pharmaceutical compositions are provided herein for this purpose.

### BRIEF DESCRIPTION OF THE DRAWINGS

FIG. 1 is a graph of intensity (%) versus diameter (nanometers) illustrating the size distribution of a paclitaxel-heparin-FA conjugate as measured by dynamic light scattering.

FIG. 2 is a bar graph illustrating observed anti Factor Xa heparin activity (IU/mg) for free heparin and a paclitaxel-heparin-FA conjugate.

FIG. 3 is a graph illustrating the biological activity of paclitaxel and a heparin-paclitaxel conjugate in promoting tubulin polymerization.

FIG. 4 is a bar graph recording cell viability of MCF-10A cells (normal breast cells) after a 48 hour incubation with paclitaxel, folic acid and a paclitaxel-heparin-FA conjugate.

FIG. 5 is a bar graph recording cell viability of KB cells (cancer cells) 48 hours after a 48 hour incubation with paclitaxel, folic acid and a paclitaxel-heparin-FA.

FIG. 6A is a graph of tumor volume (mm³) versus time (days) for mice having implanted human KB tumors being treated with saline, paclitaxel or a FA-PG-paclitaxel conjugate.

FIG. 6B is a graph of body weight (grams) versus time (days) for the mice of FIG. 6A.

FIG. 7A is a graph of tumor volume (mm³) versus time (days) for mice having implanted human KB tumors being treated with PG-paclitaxel and folic acid compared with mice being treated with a FA-PG-paclitaxel conjugate.

FIG. 7B is a graph of body weight (grams) versus time (days) for mice having implanted tumors and being treated with saline, paclitaxel or a FA-PG-paclitaxel conjugate.

FIG. 8 is a bar graph quantifying the observed amount of angiogenesis (PECAM positive area/tumor area) for implanted tumors in mice treated with saline, paclitaxel, PG-paclitaxel and folic acid or a FA-PG-paclitaxel conjugate.

FIG. 9A is a graph recording implanted tumor volume (mm³) versus time (days) in mice being treated with saline, paclitaxel or a FA-heparin-paclitaxel conjugate.

FIG. 9B is a graph recording body weight (grams) versus time (days) of the mice of FIG. 9A.

FIG. 10A is a bar graph recording observed cell viability of cells incubated with varying concentrations of paclitaxel or a FA-heparin-paclitaxel conjugate.

FIG. 10B is a graph recording body weight (grams) versus time (days) of mice having implanted tumors being treated with saline, paclitaxel or a FA-heparin-paclitaxel conjugate.

FIG. 11A is a graph of tumor volume (mm³) versus time (days) for implanted tumors in mice being treated with a FA-PG-paclitaxel conjugate or a FA-heparin-paclitaxel conjugate.

FIG. 11B is a graph of tumor volume (mm³) versus time (days) for implanted tumors in mice receiving different amounts of a FA-heparin-paclitaxel conjugate.

FIG. 11C is a graph of tumor volume (mm³) versus time (days) for implanted tumors in mice receiving either heparin-paclitaxel and folic acid or a FA-heparin-paclitaxel conjugate.

FIG. 12 is a bar graph quantifying the observed amount of angiogenesis (PECAM positive area/tumor area) for implanted tumors in mice treated with saline, paclitaxel, heparin-paclitaxel and folic acid or a FA-heparin-paclitaxel conjugate.

FIG. 13A is a graph of tumor volumes (mm³) versus time (days) for implanted KB tumors received the folate targeted drug or Abraxane™ (Taxol-bound albumin nanoparticles) at the dosage of 80 mg Taxol equivalent per kg body weight.

FIG. 13B is a comparison of the drug release profiles for Abraxane (Abx) and taxol-heparin-folic acid (THF) nanoparticles in phosphate buffered saline (PBS), in fetal calf serum (FCS), and in the presence of a protease enzyme (Cathepsin D).

FIG. 14 THF 80 denotes taxol-heparin-folic acid conjugate at 80 mg/kg dosage; TH80+FA denotes taxol-heparin at 80 mg/kg plus a physical mixture with folic acid.

### DETAILED DESCRIPTION

The following explanations of terms and methods are provided to better describe the present compounds, compositions and methods, and to guide those of ordinary skill in the art in the practice of the present disclosure. It is also to be understood that the terminology used in the disclosure is for the purpose of describing particular embodiments and examples only and is not intended to be limiting.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

In this specification and in the claims which follow, reference will be made to a number of terms which shall be understood to have the following meanings:

“Optional” or “optionally” means that the subsequently described event or circumstance can but need not occur, and that the description includes instances where said event or circumstance occurs and instances where it does not.

The term “antibody” means an immunoglobulin, whether natural or wholly or partially synthetically produced. All derivatives thereof which maintain specific binding ability are also included in the term. The term also covers any protein having a binding domain which is homologous or largely homologous to an immunoglobulin binding domain. These proteins may be derived from natural sources, or partly or wholly synthetically produced. Antibodies used herein may be monoclonal or polyclonal.

The term “antibody fragment” refers to any derivative of an antibody which is less than full-length. In an exemplary embodiment, the antibody fragment retains at least a significant portion of the full-length antibody's specific binding ability. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 50 amino acids and more typically will comprise at least about 200 amino acids.

“Atherosclerosis” refers to the progressive narrowing and hardening of a blood vessel over time. Atherosclerosis is a common form of arteriosclerosis in which deposits of yellowish plaques (atheromas) containing cholesterol, lipoid material, and lipophages are formed within the intima and inner media of large and medium-sized arteries.

“Derivative” refers to a compound or portion of a compound that is derived from or is theoretically derivable from a parent compound.

“Physiologically labile bond” refers to a bond that may be cleaved under physiological conditions (for example metabolically, solvolytically or in another manner). Such bonds are well known in the art and examples are described in Drugs of Today, Volume 19, Number 9, 1983, pp 499-538 and in Topics in Chemistry, Chapter 31, pp 306-316 and in “Design of Prodrugs” by H. Bundgaard, Elsevier, 1985, Chapter 1 (these disclosures are incorporated herein by reference).

As used herein the term “physiological conditions” refers to temperature, pH, ionic strength, viscosity, and like biochemical parameters which are compatible with a viable organism, and/or which typically exist intracellularly in a viable mammalian cell.

As used herein, the term “self-assembled” refers to any non-covalent association of two or more molecules. Typically, self-assembly occurs in an aqueous solvent, such as under physiological conditions. Examples of self-assembled structures include, without limitation, micelles and liposomes.

The term “subject” includes both human and veterinary subjects.

The term “treating a disease” refers to inhibiting the full development of a disease or condition, for example, in a subject who is at risk for a disease such as a tumor (for example, a leukemia or a lymphoma). “Treatment” refers to a therapeutic intervention that ameliorates a sign or symptom of a disease or pathological condition after it has begun to develop. As used herein, the term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of metastases, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology.

“Neoplasia” refers to the process of abnormal and uncontrolled cell growth. Neoplasia is one example of a proliferative disorder. The product of neoplasia is a neoplasm (a tumor), which is an abnormal growth of tissue that results from excessive cell division. A tumor that does not metastasize is referred to as “benign.” A tumor that invades the surrounding tissue and/or can metastasize is referred to as “malignant.” Examples of hematological tumors include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, bladder carcinoma, and CNS tumors (such as a glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma).

Water soluble polyaminoacids, include, without limitation, polylysine, polyglutamic acid, polyaspartic acid, copolymers of lysine, glutamatic acid and aspartic acid, and the like. Polyaminoacids can include the D-, L-, or both forms of the amino acid. For example, “polyglutamic acid” refers to poly-D-glutamic acid, poly-L-glutamic acid, or poly-D,L-glutamic acid. Polyglutamic acid may be abbreviated herein as “PG.”

Unless otherwise explained, all technical and scientific terms used herein have the same meaning as commonly understood by one of ordinary skill in the art to which this disclosure belongs. The singular terms “a”, “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.”

All publications, patent applications, patents, and other references mentioned herein are incorporated by reference in their entirety. In case of conflict, the present specification, including explanations of terms, will control. In addition, the materials, methods, and examples are illustrative only and not intended to be limiting.

Disclosed herein are conjugates, compounds, compositions and methods for treating particular tissues, particularly hyper-proliferative tissues, selectively. Generally the conjugates include at least three components, namely a chemotherapeutic agent or an imaging agent a nanocarrier that is a nanoparticle, an organic polymer or both; and a targeting agent. The three components can be covalently or non-covalently associated.

In one embodiment, the components of the disclosed conjugates are covalently linked to form nanocarrier conjugate compounds. Examples of such compounds include ternary or tripartite molecules including at least one nanocarrier, targeting agent and therapeutic agent. In one embodiment the disclosed compounds have one of the following formulas:

A-X—Y

X-A-Y

or

X—Y-A

With respect to the general formulas, A represents a chemotherapeutic agent or an imaging agent; X represents a nanocarrier that is a nanoparticle, an organic polymer or both; and Y represents a targeting agent. Higher order compounds also are disclosed, including quaternary conjugate compounds.

Reference will now be made in detail to the presently preferred embodiments of the disclosed conjugates compounds, compositions and methods.

I. Nanocarriers

The nanocarrier component of the conjugates and compounds disclosed herein can function to present a multivalent display of the therapeutic agent, the targeting agent, or both. In another aspect, the nanocarrier component may confer sufficient size upon the compounds that the compounds benefit from the EPR effect.

Typically, “nanocarrier” refers to a nanoparticle having a diameter of less than about 1500 nanometers or an organic polymer having a molecular weight of more than about 1000 daltons. As used herein the term “nanoparticle” refers to a particle having a diameter of from about 1 nanometer to about 1500 nanometers. Exemplary types of nanoparticles include, without limitation, colloidal and non-colloidal metal clusters and polymeric nanoparticles, such as micelles, liposomes and oil-in-water emulsions. Typically, the nanoparticles employed herein range in diameter from about 1 nanometer to about 1200 nanometers and more typically from about 10 to about 400 nanometers, such as from about 100 to about 150 nanometers. However, certain nanoparticles, for example metal clusters, such as gold clusters can have a diameter as small as about 0.7 nanometers. In certain embodiments a nanoparticle size of less than 400 nanometers, and even less than 150 nanometers, such as about 100 nanometers provides the cell targeting for the present compounds.

In principle any nanoparticle, organic polymer or material that assembles to form a nanoparticle is suitable for use as the nanocarrier component of the compounds described herein. In one embodiment, wherein the nanocarrier component is an organic polymer, the polymer self assembles under physiological conditions to form a self-assembled nanoparticle that can include a plurality or aggregation of the organic polymer-containing molecules described herein. However, such self-assembled nanoparticles also can be formed under non-physiological conditions provided that the nanoparticles do not disassemble under physiological conditions. In one embodiment, the organic polymer is a polyionic polymer, such as a polycationic or polyanionic polymer. In one embodiment the polymer includes a water soluble polyaminoacid, a water soluble polysaccharide or both. Examples of cationic polymers that can be used in the invention include, but are not limited to, DEAE dextran (diethyleneaminoethyl dextran), polyethyleneimine (PEI), chitin, chitosan (D-acetylated chitin) and polyamino acids with a positive charge, such as polylysine. Examples of anionic polymers that can be used in the present invention include, but are not limited to, dextran sulfate, mucopolysaccharides, such as heparin, hyaluronic acid, and polyamino acids having a negative charge. Gel forming anionic, such as alginate and carageenan polymers also can be used. Additional charged and uncharged organic polymers that can be used as nanocarriers include, without limitation, N-(2-hydroxypropyl)-methacrylamide copolymer, poly-2-hydroxyethylmethacrylate, poly(poly-2-hydroxyethylmethacrylate co-methacrylate), polystyrene, polyethylene glycol, polyoxamers, polyoxamines, poly(methyl methacrylate), poly(butyl 2-cyanoacrylate), dextran, carboxymethyl dextran, cellulose, carboxymethyl cellulose and chitosan. In exemplary embodiments the nanocarrier comprises polyanionic materials, such as heparin or polyglutamic acid.

In one embodiment, nanocarrier conjugate compounds include, in addition to a targeting agent, a hydrophilic nanocarrier, such as a polycationic or polyanionic polymer, and a hydrophobic component, such as a hydrophobic chemotherapeutic or imaging agent. Such compounds are amphiphilic, which promotes self-assembly of the conjugate compounds. One embodiment of such an amphiphilic compound having a hydrophobic chemotherapeutic agent is a paclitaxel-functionalized heparin polymer. Another embodiment of such an amphiphilic compound is an epothilone-functionalized heparin polymer. In particular embodiments, these compounds include folate or a folate derivative as the targeting agent.

In one embodiment the disclosed compounds effectively act as prodrugs by releasing the therapeutic agent at a particular targeted tissue or cell. For example, a therapeutic agent can be bound to the nanocarrier or targeting agent via a physiologically labile bond, such as an ester bond, that is cleaved at the target. In one embodiment, the conjugate compound is relatively benign, whereas the therapeutic agent is cytotoxic when not included in a conjugate compound.

The nanoparticles and organic polymers suitable for use in the presently disclosed conjugates can be monodisperse or polydisperse. Typically, when the conjugate includes an organic polymer, the organic polymer is polydisperse. Generally suitable examples of polymers have an average molecular weight of at least about 1,000 daltons. Such polymers typically have an average molecular weight of from about 1,000 to about 150,000 daltons. More typically suitable polymers have a molecular weight of from about 5,000 to about 100,000 daltons, such as from about 10,000 to about 50,000 daltons.

The nanocarrier component can contain plural functional groups for derivatization with therapeutic agents, imaging agents and targeting agents. Thus, in one embodiment of the conjugate compounds, the compounds have a formula

Aₘ-X-Yₙ

X-Aₘ-Yₙ

or

X—Yₙ-Aₘ

wherein A, X and Y are as described above and n and m independently are integers from 1 to about 500, such as from 5 to about 150. Typically, n and m are integers of from 1 to about 50. In exemplary embodiments the sum of m and n is from about 10 to about 100, such as about 50. In compounds having n greater than one, the targeting agents can be the same or different. Similarly, in compounds wherein m is greater than one, the therapeutic agents can be the same or different. An exemplary embodiment of a compound disclosed herein has the formula Aₘ-X-Yₙ, wherein A is a paclitaxel moiety, X is heparin, Y is a folate moiety and n and m independently are from 1 to about 50. One example of such an embodiment is represented by the structure

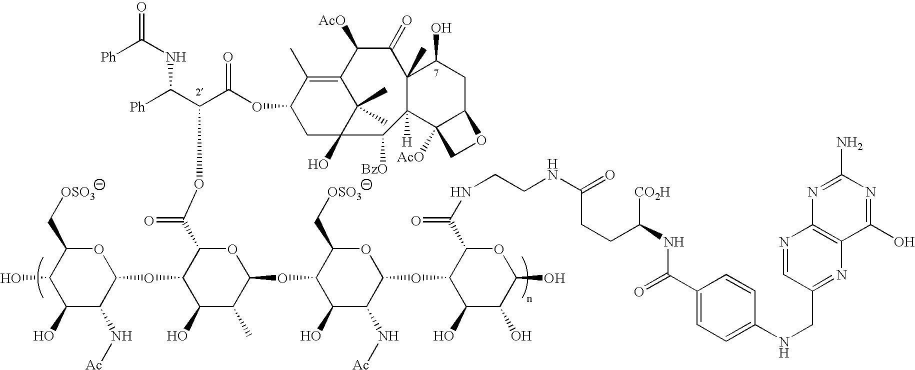

Another exemplary embodiment of a compound disclosed herein has the formula Aₘ-X-Yₙ, wherein A is an epothilone moiety, X is heparin, Y is a folate moiety and n and m independently are from 1 to about 50. One such embodiment may be represented by the structure

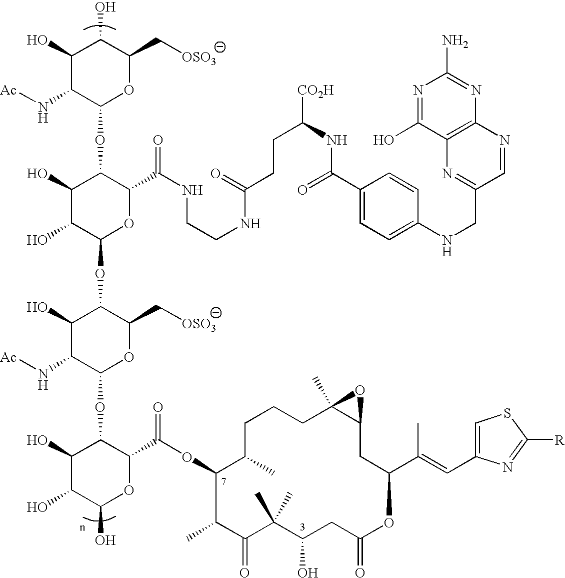

In another exemplary embodiment, the disclosed compounds include those having the formula

Aₘ-X-Yₙ

wherein A, X and Y are as described above and m and n independently are from 1 to 10, such as from 1 to 5. In a particular embodiment, m is 2 and n is 1. In this embodiment the A moieties can be the same or different. In another embodiment m is 1 and n is 2 and the Y moieties can be the same or different. In these embodiments, the compounds are quaternary compounds including a nanocarrier an imaging or therapeutic agent and two targeting agents. Alternatively, quaternary compounds can include a nanocarrier, a targeting agent and two A groups, such as two therapeutic agents, two imaging agents or a therapeutic agent and an imaging agent.

Examples of the presently disclosed conjugate compounds, for example ternary and quaternary conjugates, can be represented schematically as

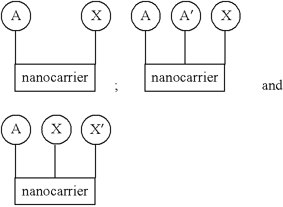

wherein A and X are as described above, A and A′ represent different chemotherapeutic or imaging agents, and X and X′ represent different targeting agents. In one embodiment of a ternary nanocarrier conjugate, the nanocarrier is functionalized with plural copies of A and/or X. Similarly, embodiments of quaternary nanocarrier conjugates are functionalized with plural copies of A, A′, X and/or X′. Certain examples of quaternary nanocarrier conjugates include at least one chemotherapeutic agent and at least one imaging agent.

In one embodiment, the nanocarrier is a nanoparticle, such as a metal cluster, examples of which include clusters comprising Ag, Au, Pt, Pd, Co, Fe or mixtures thereof. As noted above, examples of such metal clusters may be either colloidal or non-colloidal materials. Certain suitable metal clusters also can include alloys of different metals. In one embodiment the metal is magnetic, and as such can be used for imaging. Typically the metal nanoparticles are paramagnetic. Examples of such nanoparticles include iron oxide nanoparticles comprising Fe₂O₃, Fe₃O₄ or both.

A variety of methods suitable for preparing examples of the presently disclosed compounds have been developed for assembling and functionalizing metal clusters. These methods have focused on the use of covalent linker molecules that possess functionalities at opposing ends with chemical affinities for the colloids of interest. Examples of this approach are described by Brust et al. Adv. Mater. 1995 7, 795-797, involves the use of gold colloids and well-established thiol adsorption chemistry, Bain & Whitesides, Angew. Chem. Int. Ed. Engl. 1989, 28, 506-512 and Dubois & Nuzzo, Annu. Rev. Phys. Chem. 1992, 43, 437-464. U.S. Pat. No. 6,767,702 to Mirkin et al. and U.S. Application Publication No. 2003/0077625, both of which are incorporated herein by reference, disclose metal clusters and methods and reagents for functionalizing such clusters that can be used to prepare the presently disclosed conjugate compounds.

II. Therapeutic Agents and Imaging Agents

Any therapeutic agent can be used in the disclosed compounds and methods. Appropriate therapeutic agents can be selected based upon the particular tissue or cell type being targeted. That is, the choice of a particular therapeutic agent depends on the particular target molecule or cell and the biological effect is desired to evoke. For example, in one embodiment the compounds include a targeting agent that targets hyper-proliferative cells, such as cancer cells. In such embodiments the therapeutic agent can be an anti-proliferative agent, including any chemical agent with therapeutic usefulness in the treatment of proliferative diseases or diseases characterized by abnormal cell growth. Such diseases include tumors, neoplasms, and cancer as well as diseases characterized by hyperplastic growth such as psoriasis. In one embodiment, an anti-proliferative agent is an agent of use in treating a lymphoma, leukemia, or another tumor. In one embodiment, an anti-proliferative agent is a radioactive compound. One of skill in the art can readily identify an anti-proliferative agent of use (e.g. see Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2nd ed., 2000 Churchill Livingstone, Inc; Baltzer, L., Berkery, R. (eds): Oncology Pocket Guide to Chemotherapy, 2nd ed. St. Louis, Mosby-Year Book, 1995; Fischer, D. S., Knobf, M. F., Durivage, H. J. (eds): The Cancer Chemotherapy Handbook, 4th ed. St. Louis, Mosby-Year Book, 1993).

Classes of useful anti-proliferative agents that can be used in the present compounds include, without limitation, microtubule binding agent, a toxin, a DNA intercalator or cross-linker, a DNA synthesis inhibitor, a DNA and/or RNA transcription inhibitor, an enzyme inhibitor, a gene regulator, enediyne antibiotics and/or an angiogenesis inhibitor. In one embodiment the molecules have sufficient selectivity for a hyper-proliferative tissue such that therapeutic agents having a higher cytotoxicity than is normally acceptable can be used.

“Microtubule binding agent” refers to an agent that interacts with tubulin to stabilize or destabilize microtubule formation thereby inhibiting cell division. Suitable microtubule binding agents include, without limitation, paclitaxel, docetaxel, vinblastine, vindesine, vinorelbine (navelbine), the epothilones, colchicine, dolastatin 15, nocodazole, podophyllotoxin and rhizoxin. Analogs and derivatives of such compounds also can be used and will be known to those of ordinary skill in the art. For example, suitable epothilones and epothilone analogs for incorporation into the present compounds are described in International Publication No. WO 2004/018478, which is incorporated herein by reference. Taxoids, such as paclitaxel and docetaxel are currently believed to be particularly useful as therapeutic agents in the presently disclosed compounds. Examples of additional useful taxoids, including analogs of paclitaxel are taught by U.S. Pat. Nos. 6,610,860 to Holton, 5,530,020 to Gurram et al. and 5,912,264 to Wittman et al. Each of these patents is incorporated herein by reference.

The therapeutic agent may be a cytotoxin which is used to bring about the death of a particular target cell. Exemplary toxins include Pseudomonas exotoxin (PE), ricin, abrin, diphtheria toxin and subunits thereof, ribotoxin, ribonuclease, saporin, as well as botulinum toxins A through F. These toxins are well known in the art and many are readily available from commercial sources (for example, Sigma Chemical Company, St. Louis, Mo.).

One example of a suitable therapeutic agent, diphtheria toxin, is isolated from Corynebacterium diphtheriae. Typically, diphtheria toxin for use in immunotoxins is mutated to reduce or to eliminate non-specific toxicity. A mutant known as CRM107, which has full enzymatic activity but markedly reduced non-specific toxicity, has been known since the 1970's (Laird and Groman, J. Virol. 1976, 19, 220), and has been used in human clinical trials. See, U.S. Pat. No. 5,792,458 and U.S. Pat. No. 5,208,021. As used herein, the term “diphtheria toxin” refers as appropriate to native diphtheria toxin or to diphtheria toxin that retains enzymatic activity but which has been modified to reduce non-specific toxicity.

Another exemplary toxin, suitable for use as the therapeutic component of the presently disclosed conjugates is ricin. Ricin is a lectin isolated from Ricinus communis (castor bean). The term “ricin” also references toxic variants thereof. For example, see, U.S. Pat. No. 5,079,163 and U.S. Pat. No. 4,689,401. Ricinus communis agglutinin (RCA) occurs in two forms designated RCA60 and RCA120 according to their molecular weights of approximately 65 and 120 kD, respectively (Nicholson & Blaustein, J. Biochim. Biophys. Acta 1972, 266, 543). The A chain is responsible for inactivating protein synthesis and killing cells. The B chain binds ricin to cell-surface galactose residues and facilitates transport of the A chain into the cytosol (Olsnes et al. Nature 1974, 249, 627-631, and U.S. Pat. No. 3,060,165). Another toxic lectin, abrin, includes toxic lectins from Abrus precatorius. The toxic principles, abrin a, b, c, and d, have a molecular weight of from about 63 and 67 kD and are composed of two disulfide-linked polypeptide chains A and B. The A chain inhibits protein synthesis; the B-chain (abrin-b) binds to D-galactose residues (see, Funatsu, et al. Agr. Biol. Chem. 1988, 52, 1095; and Olsnes, Methods Enzymol. 1978, 50, 330-335,).

In one embodiment, a toxin used to terminate a targeted cell is Pseudomonas exotoxin (PE). Native Pseudomonas exotoxin A (“PE”) is an extremely active monomeric protein (molecular weight 66 kD), secreted by Pseudomonas aeruginosa, which inhibits protein synthesis in eukaryotic cells. The native PE sequence and the sequence of modified PE is provided in U.S. Pat. No. 5,602,095, which is incorporated herein by reference. The method of action of PE is inactivation of the ADP-ribosylation of elongation factor 2 (EF-2). The exotoxin contains three structural domains that act in concert to cause cytotoxicity. Domain Ia (amino acids 1-252) mediates cell binding. Domain II (amino acids 253-364) is responsible for translocation into the cytosol and domain III (amino acids 400-613) mediates ADP ribosylation of elongation factor 2. The function of domain Ib (amino acids 365-399) remains undefined, although a large part of it, amino acids 365-380, can be deleted without loss of cytotoxicity. See, Siegall et al. J. Biol. Chem. 1989, 264, 14256-14261.

The term “Pseudomonas exotoxin” (“PE”) as used herein refers as appropriate to a full-length native (naturally occurring) PE or to a PE that has been modified. Such modifications may include, but are not limited to, elimination of domain Ia, various amino acid deletions in domains Ib, II and III, single amino acid substitutions and the addition of one or more sequences at the carboxyl terminus. See, Siegall et al., supra. In several examples, the cytotoxic fragment of PE retains at least 50%, preferably 75%, more preferably at least 90%, and most preferably 95% of the cytotoxicity of native PE. In one embodiment, the cytotoxic fragment is more toxic than native PE.

Thus, the PE used in the targeted conjugates disclosed herein includes the native sequence, cytotoxic fragments of the native sequence, and conservatively modified variants of native PE and its cytotoxic fragments. Cytotoxic fragments of PE include those which are cytotoxic with or without subsequent proteolytic or other processing in the target cell (e.g., as a protein or pre-protein). Cytotoxic fragments of PE known in the art include PE40, PE38, and PE35.

In several embodiments, the PE has been modified to reduce or eliminate non-specific cell binding, typically by deleting domain Ia, as taught in U.S. Pat. No. 4,892,827, although this can also be achieved, for example, by mutating certain residues of domain Ia. U.S. Pat. No. 5,512,658, for instance, discloses that a mutated PE in which Domain Ia is present but in which the basic residues of domain Ia at positions 57, 246, 247, and 249 are replaced with acidic residues (glutamic acid, or “E”) exhibits greatly diminished non-specific cytotoxicity. This mutant form of PE is sometimes referred to as PE4E.

PE40 is a truncated derivative of PE (see, Pai et al. Proc. Nat'l Acad. Sci. USA 1991, 88, 3358-62; and Kondo et al. J. Biol. Chem. 1988, 263, 9470-9475). PE35 is a 35 kD carboxyl-terminal fragment of PE in which amino acid residues 1-279 have deleted and the molecule commences with a met at position 280 followed by amino acids 281-364 and 381-613 of native PE. PE35 and PE40 are disclosed, for example, in U.S. Pat. No. 5,602,095 and U.S. Pat. No. 4,892,827. In some embodiments, the cytotoxic fragment PE38 is employed. PE38 is a truncated PE pro-protein composed of amino acids 253-364 and 381-613 of which is activated to its cytotoxic form upon processing within a cell (see e.g., U.S. Pat. No. 5,608,039, and Pastan et al., Biochim. Biophys. Acta 1333:C1-C6, 1997). While in some embodiments, the PE is PE4E, PE40, or PE38, any form of PE in which non-specific cytotoxicity has been eliminated or reduced to levels in which significant toxicity to non-targeted cells does not occur can be used in the immunotoxins disclosed herein so long as it remains capable of translocation and EF-2 ribosylation in a targeted cell.

Conservatively modified variants of PE or cytotoxic fragments thereof have at least 80% sequence similarity, preferably at least 85% sequence similarity, more preferably at least 90% sequence similarity, and most preferably at least 95% sequence similarity at the amino acid level, with the PE of interest, such as PE38.

Ribonucleases also can be used as toxins in the disclosed, targeted conjugate compounds (see, Suzuki et al. Nat. Biotech. 1999, 17, 265-70). Exemplary ribotoxins such as α-sarcin and restrictocin are discussed in, for example, Rathore et al. Gene 1997, 190, 31-35; and Goyal and Batra, Biochem. 2000, 345 Pt 2 247-54.

DNA intercalators and cross-linking agents that can be incorporated into the disclosed compounds include, without limitation, cisplatin, carboplatin, oxaliplatin, mitomycins, such as mitomycin C, bleomycin, chlorambucil, cyclophosphamide and derivatives and analogs thereof.

DNA synthesis inhibitors suitable for use as therapeutic agents include, without limitation, methotrexate, 5-fluoro-5′-deoxyuridine, 5-fluorouracil and analogs thereof.

Examples of suitable enzyme inhibitors for use in the presently disclosed conjugates include, without limitation, camptothecin, etoposide, formestane, trichostatin and derivatives and analogs thereof.

Suitable therapeutics that affect gene regulation include agents that result in increased or decreased expression of one or more genes, such as, without limitation, raloxifene, 5-azacytidine, 5-aza-2′-deoxycytidine, tamoxifen, 4-hydroxytamoxifen, mifepristone and derivatives and analogs thereof.

Suitable enediyne antibiotics for incorporation into the disclosed conjugates include naturally occurring enediyne-containing compounds, non-natural compounds and derivatives thereof. Naturally-occurring enediyne antibiotics, including dynemicin (Konishi, et al. J. Chem. Soc. 1990, 112, 3715-3716, esperamicin (Golik et al. J. Amer. Chem. Soc. 1987 109, 3462-3464) and calicheamicin (Lee et al. J. Amer. Chem. Soc. 1987, 109, 3464-3466) are very potent cytotoxins, with IC₅₀ values for inhibition of growth of tumor cell cultures in the low picomolar range. Examples of non-natural enediyne antibiotic compounds suitable for incorporation into the presently disclosed conjugates are disclosed by Nicolaou, K. C. et al, Science 1992, 256, 1172-1178; Nicolaou et al. Proc. Natl. Acad. Sci. USA. 1993, 90, 5881-5888; J. Amer. Chem. Soc. 1992, 114, 8890-8907; J. Amer. Chem. Soc. 1993, 115, 7944-7953; J. Amer. Chem. Soc. 1992, 114, 8908-8921; Wender et al. J. Org. Chem. 1993, 58, 5867-5869; Synthesis, 1994, 1279-1282; U.S. Pat. Nos. 6,124,310 to Denny et al. and 6,514,995 to Zaleski and Rawat. Each of these disclosures is incorporated herein by reference.

Suitable DNA and/or RNA transcription regulators, including, without limitation, actinomycin D, daunorubicin, doxorubicin and derivatives and analogs thereof also are suitable for use in the presently disclosed compounds.

The term “angiogenesis inhibitor” is used herein, to mean a molecule including, but not limited to, biomolecules, such as peptides, proteins, enzymes, polysaccharides, oligonucleotides, DNA, RNA, recombinant vectors, and small molecules that function to inhibit blood vessel growth. Angiogenesis is implicated in certain pathological processes, such as those involved in disorders such as diabetic retinopathy, chronic inflammatory diseases, rheumatoid arthritis, dermatitis, psoriasis, stomach ulcers, and most types of human solid tumors.

Angiogenesis inhibitors are known in the art and examples of suitable angiogenesis inhibitors include, without limitation, angiostatin K1-3, staurosporine, genistein, fumagillin, medroxyprogesterone, suramin, interferon-alpha, metalloproteinase inhibitors, platelet factor 4, somatostatin, thromobospondin, endostatin, thalidomide, and derivatives and analogs thereof.

Other therapeutic agents, particularly anti-tumor agents, that may or may not fall under one or more of the classifications above, also are suitable for incorporation into the presently disclosed compounds. By way of example, such agents include adriamycin, apigenin, rapamycin, zebularine, cimetidine, and derivatives and analogs thereof.

In certain embodiments, the compounds are targeted to atherosclerotic lesions. In such embodiments the therapeutic agent is effective to reduce or prevent lipid accumulation by the vessel, to increase plaque stability of an atherosclerotic lesion, to inhibit atherosclerotic lesion formation or development, or to induce atherosclerotic lesion regression. Examples of suitable therapeutic agents include those taught by U.S. Pat. No. 6,734,208, to Grainger et al., which is incorporated herein by reference.

Additional suitable therapeutic agents include anti-sense oligonucleotides and the like; biological response modifiers such as muramylpeptides; antifungal agents such as ketoconazole, nystatin, griseofulvin, flucytosine, miconazole or amphotericin B; hormones or hormone analogues such as growth hormone, melanocyte stimulating hormone, estradiol, beclomethasone dipropionate, betamethasone, cortisone acetate, dexamethasone, flunisolide, hydrocortisone, methylprednisolone, paramethasone acetate, prednisolone, prednisone, triamcinolone or fludrocortisone acetate; vitamins such as cyanocobalamin or retinoids; enzymes such as alkaline phosphatase or manganese superoxide dismutase; antiallergic agents such as amelexanox; inhibitors of tissue factor such as monoclonal antibodies and Fab fragments thereof, synthetic peptides, nonpeptides and compounds downregulating tissue factor expression; inhibitors of platelets such as, GPIa, GPIb and GPIIb-IIIa, ADP receptors, thrombin receptors, von Willebrand factor, prostaglandins, aspirin, ticlopidin, clopigogrel and reopro; circulatory drugs such as propranolol; metabolic potentiators such as glutathione; antituberculars such as p-aminosalicylic acid, isoniazid, capreomycin sulfate, cyclosexine, ethambutol, ethionamide, pyrazinamide, rifampin or streptomycin sulphate; antivirals such as acyclovir, amantadine, azidothymidine, ribavirin or vidarabine; blood vessel dilating agents such as diltiazem, nifedipine, verapamil, erythritol tetranitrate, isosorbide dinitrate, nitroglycerin or pentaerythitol tetranitrate; antibiotics such as dapsone, chloramphenicol, neomycin, cefaclor, cefadroxil, cephalexin, cephradine, erythromycin, clindamycin, lincomycin, amoxicillin, ampicillin, bacampicillin, carbenicillin, dicloxacillin, cyclacillin, picloxacillin, hetacillin, methicillin, nafcillin, penicillin, polymyxin or tetracycline; antiinflammatories such as diflunisal, ibuprofen, indomethacin, meclefenamate, mefenamic acid, naproxen, phenylbutazone, piroxicam, tolmetin, aspirin or salicylates; antiprotozoans such as chloroquine, metronidazole, quinine or meglumine antimonate; antirheumatics such as penicillamine; narcotics such as paregoric; opiates such as codeine, morphine and analogs thereof; cardiac glycosides such as deslaneside, digitoxin, digoxin, digitalin or digitalis; neuromuscular blockers such as atracurium mesylate, gallamine triethiodide, hexafluorenium bromide, metocurine iodide, pancuronium bromide, succinylcholine chloride, tubocurarine chloride or vecuronium bromide; sedatives such as amobarbital, amobarbital sodium, apropbarbital, butabarbital sodium, chloral hydrate, ethchlorvynol, ethinamate, flurazepam hydrochloride, glutethimide, methotrimeprazine hydrochloride, methyprylon, midazolam hydrochloride, paraldehyde, pentobarbital, secobarbital sodium, talbutal, temazepam or triazolam.

Additional therapeutic agents may be selected for incorporation into the disclosed compounds as will be apparent to those of ordinary skill in the art upon review of the present specification.

In one embodiment, the compounds disclosed herein can include an imaging agent. As used herein, the term “imaging agent” refers to compounds that can be detected. Examples of imaging agents include magnetic resonance imaging contrast agents, computed tomography (CT scan) imaging agents, optical imaging agents and radioisotopes. In certain compounds according to this embodiment, an imaging agent optionally may be used in place of a therapeutic agent. Thus, the presently disclosed compounds can be used to image a targeted tissue selectively.

Particular examples of suitable imaging agents include, without limitation gadolinium chelating agents, such as gadolinium-DTPA (Gd-DTPA), CT scan imaging agents, such as those including a heavy metal such as iron chelates; near-infrared optical imaging agents, such as Cy 5.5, indocyanine green (ICG) and its derivatives, and the radionuclides indium-111, technetium-99m, yttrium-90 and holmium-166. Additionally, positron emission tomography (PET) may be possible using positron emitters of oxygen, nitrogen, iron, carbon, or gallium.

In one embodiment, the disclosed compounds include those having both a therapeutic agent and an imaging agent. One example of such compounds has the formula

Aₘ-X-Yₙ

wherein m is 2 or greater, such as from 2 to about 500, such as from 5 to about 150. Typically m is from 2 to about 50, such as from 2 to about 10. Examples of such compounds can include a single imaging agent and a plurality of therapeutic agents or a single therapeutic agent and a plurality of imaging agents. However, in one embodiment, m is 2 and the formula includes a single therapeutic agent and a single imaging agent.

III. Targeting Agents

The targeting agent can be any ligand moiety, such as an antibody, growth factors, cytokines, cell adhesion molecules, their receptors, peptide, protein or small molecule, such as a receptor agonist, antagonist or enzyme inhibitor that binds to a cell, typically a particular cellular receptor. It is understood that when a particular targeting agent is referred to, fragments, residues and derivatives thereof also are intended.

In one embodiment, the compounds include plural targeting agents, which can be the same or different. For example, a compound can present an effectively multivalent display of plural targeting agents, to enhance affinity, avidity or selectivity of a nanocarrier therapeutic. Alternatively, the disclosed compounds can include different targeting agents to, for example target different cell-types or tissues. Specifically, the compounds include those of the formula

Aₘ-X-Yₙ

wherein A, X, Y and m are as described above, and n is from 2 to about 500, such as from 5 to about 150. Such compounds include those having n be an integer of from 2 to about 50, such as from 2 to about 10. In one embodiment of such a compound, n is two and the formula includes two different Y moieties.

Both conventional and genetically engineered antibodies may be employed as targeting agents. The use of human antibodies may be preferred to avoid possible immune reactions. In one embodiment, the targeting agent is an antibody that binds to a member of the human epidermal growth factor receptor (EGFR) family. The human EGFR family includes EGFR-1 (HER-1), EGFR-2 (HER-2), EGFR-3 (HER-3) and EGFR 4 (HER-4). EGFR expression has been documented extensively in a wide variety of malignant tumors including lung, head and neck, colon, breast, and prostate, etc. ⁵⁰-56 Several studies, have demonstrated that overexpression of EGFR correlates with reduced overall survival, increased risk of disease recurrence and metastasis. (See, for example, Grandis J R, Melhem M F, Gooding W E, et al. Levels of TGF-α and EGFR protein in head and neck squamous cell carcinoma and patient survival. J Natl Cancer Inst 1998, 90:824-32; Mauizi M, Almadori G, Ferrandina G, et al. Prognostic significance of epidermal growth factor receptor in laryngeal squamous cell carcinoma. Br J Cancer 1996, 74:1253-7; Yamanaka Y, Friess H, Kobrin M S, Buchlen M, Beger H G, Korc M. Coexpression of epidermal growth factor receptor and ligands in human pancreatic cancer is associated with enhanced tumor aggressiveness. Anticancer Res 1993, 13:565-70; Neal D E, Sharples L, Smith K, Fennelly J, Hall R R, Hams A L. The epidermal growth factor receptor and the prognosis of bladder cancer. Cancer 1993, 65: 1619-25.) Overexpression of EGFR also correlates to a poor response to therapeutic agents. See, Aziz S A, Pervez S, Khan S, Kayani N, Rahbar M H. Epidermal growth factor receptor (EGFR) as a prognostic marker: an immunohistochemical study on 315 consecutive breast carcinoma patients. J Pak Med Assoc 2002, 52:104-10; Tsutsui S, Ohno S, Murakami S, Hachitanda Y, Oda S. Prognostic value of epidermal growth factor receptor (EGFR) and its relationship to the estrogen receptor status in 1029 patients with breast cancer. Breast Cancer Res Treat 2002, 71:67-75; Nicholson R I, Gee J M, Harper M E. EGFR and cancer prognosis. Eur J Cancer 2001, 37 Suppl 4:S9-15. In a particular embodiment, a single chain EGFR antibody (ScFv EGFR) is used as a targeting agent for the formulation of nanocarrier conjugates disclosed herein.

In one embodiment, the targeting agent is a ligand for a cell surface receptor. In one aspect of this embodiment the targeting agent induces receptor-mediated endocytosis, such as potocytosis. Examples of suitable targeting agents that may induce receptor-mediated endocytosis include, without limitation, folate, insulin, nerve growth factor, luteinizing hormone, calcitonin and catecholamines.

In one embodiment of the compounds disclosed herein, the targeting agent selected binds to a receptor that is present at higher density on the targeted cells. For example, certain tumor cells over-express receptors involved in the uptake of folate, biotin and/or vitamins, such as vitamin B₁₂. Other receptors that can be targeted on tumor cells include, without limitation, transferrin receptor, mucins, multiple P-glycoprotein, cathepsin B and CD44. Accordingly, disclosed herein are compounds that employ antibodies directed to the receptors recited above, as well as compounds that employ small molecule targeting agents, such as folate, vitamin B₁₂ and/or biotin, and derivatives thereof as targeting agents to direct therapeutic agents to such cells. The folate receptor, for example, is known to be overexpressed on the surface of cancer cells in the case of epithelial malignancies, such as ovarian, colorectal, and breast cancer, whereas in most normal tissue it is expressed in very low levels. See, Leamon and Reddy Adv. Drug Deliv. Rev. 2004, 56, 1127-1141; Lee and Low J. Biol. Chem. 1994, 269, 3198-3204. Embodiments of targeting agents directed to the folate receptor include, without limitation, folic acid, folic acid derivatives and analogs, antifolates and deazafolates. As used herein, the term “folate” shall include all such structures. Examples of such folates include folic acid, dihydrofolic acid, tetrahydrofolic acid, folinic acid, pteropolyglutamic acid, dihydrofolates, tetrahydrofolates, tetrahydropterins, 1-deaza, 3-deaza, 5-deaza, 8-deaza, 10-deaza, 1,5-dideaza, 5,10-dideaza, 8,10-dideaza and 5,8-dideaza folate analogs, and antifolates.

In one embodiment a peptide targeting agent is selected using combinatorial techniques, such as phage display (see, U.S. Pat. No. 5,223,409) or variations of phage display, with which those of ordinary skill in the art will be familiar. Similarly, small molecule ligands, such as receptor agonists, antagonists and inhibitors, suitable for use as targeting agents can be prepared and selected using combinatorial techniques.

Cytokines, growth factors and peptide hormones that can be used as the targeting component in the presently disclosed compounds include epidermal growth factor, nerve growth factor, somatostatin, endothelin, interleukin-1, interleukin-2, tumor necrosis factor, parathyroid hormone, insulin like growth factor I and fragments thereof.

In one embodiment the disclosed compounds employ anti-angiogenic factors as targeting agents, such as, interferon-α, interferon-γ, thrombospondin, angiogenin, bradykinin, basic fibroblast growth factor, fibrin, fibrinogen, histamine, nicotinamide, platelet activating factor, prostaglandins, spermine, substance P, transforming growth factor-α, transforming growth factor-β, vitronectin and fragments thereof. Targeting agents also can be selected to target atherosclerotic lesions, for example, annexin V atherosclerotic plaque binding peptides such as YRALVDTLK, YAKFRETLEDTRDRMY and RALVDTEFKVKQEAGAK, can be used to target such lesions. Additional targeting agents that can be used target receptors associated with angiogenesis but may not be angiogenetic factors, such agents include, without limitation, antibodies, angiopoietin, α₂-antiplasmin, endosialin, hepatocyte growth factor, leukemia inhibitory factor, RGD-peptides, such as cyclic RGD_{D}FV, placental growth factor, selecting, pleiotropin, thyrnidine phosphorylase, tumor growth factor, sialyl Lewis X, osteopontin, syndecan, tissue factor, VCAM, vascular endothelial growth factor related protein, vascular endothelial growth factor-A receptor, von Willebrand factor-related antigen and fragments thereof.

Additional over-expressed receptors that can be targeted by using their ligands in the presently disclosed compounds and compositions are known to those of ordinary skill in the art.

IV. Conjugation Chemistry

Numerous methods and reagents for coupling the components of the presently disclosed conjugates are well known to those of ordinary skill in the art. Table 1 lists representative suitable functional groups that may be present on a polymer, nanoparticle, therapeutic agent, imaging agent, targeting agent or linker and can be used to couple these materials.

TABLE 1
Functional Group Pairs for Conjugation Chemistry

| Functional Groups: | Reacts With: |
|---|---|
| ketone groups | amino, hydrazido and aminooxy |
| (such as aldehydes) | |
| imide | amino, hydrazido and aminooxy |
| cyano | hydroxy |
| alkylating agents | thiol, amino, hydrazido, aminooxy |
| (such as haloalkyl | |
| groups and maleimide deriva- | |
| tives) carboxyl groups | amino, hydroxy, hydrazido, aminooxy |
| (including activated | |
| carboxyl groups) | |
| activated sulfonyl groups | amino, hydroxy, hydrazido, aminooxy |
| (such as sulfonyl chlorides) | |
| sulfhydryl | sulfhydryl |
| His-tag (such as a 6-His tagged | nickel nitriloacetic acid |
| peptide or protein) | |

U.S. Pat. No. 6,303,752 to Olsen et al., which is incorporated herein by reference, describes use of the functional groups listed in Table 1 among others to couple linkers, polymers and proteins.

Other reagents than the exemplary coupling partners in Table 1 can be used to couple components of the compounds disclosed herein. For example, azide-containing compounds can be coupled to other molecules via Staudinger ligation. Suitable reagents for Staudinger ligation can be prepared according to the methods disclosed by Saxon and Bertozzi in U.S. Pat. No. 6,570,040 and by Raines et al. in U.S. Patent Publication No. 20040087779. The '040 patent and the '779 publication are incorporated herein by reference in their entireties.

In particular, the coupling methods suitable for assembling the disclosed conjugates include, without limitation, amino-reactive acylating agents, such as isocyanates and isothiocyanates, which form stable urea and thiourea derivatives respectively. Examples of such compounds have been used for protein crosslinking as described by Schick, A. F. et al. in J. Biol. Chem. 1961 236, 2477. Active esters are particularly useful for preparing the disclosed conjugate compounds, such as nitrophenylesters or N-hydroxysuccinimidyl esters. Suitable reagents and conditions for acylating amino groups using active esters are described by Bodanszky, M. and Bodanszky, A.; The Practice of Peptide Synthesis; Springer Verlag, New York, 1994; and by Jones, J.; Amino Acid and Peptide Synthesis; 2nd ed.; Oxford University Press, 2002, both of which are incorporated herein by reference.

Other suitable linkages formed using the reagents listed in Table 1 include disulfide linkages, formed by the oxidative coupling of two sulfhydryl-containing molecules. Another exemplary coupling technique employs a chelated nickel moiety, such as nickel nitriloacetic acid, which couples with His-tagged peptides and proteins, including His-tagged antibodies. For example, in one embodiment, a C-terminal His-tagged antibody, such as ScFv EGFR is conjugated to a nanocarrier, such as heparin or polyglutamic acid that is derivatized with a nickel nitriloacetic acid moiety. One example of such a structure is a heparin-ScFv EGFR-Taxotere™ conjugate having the following formula:

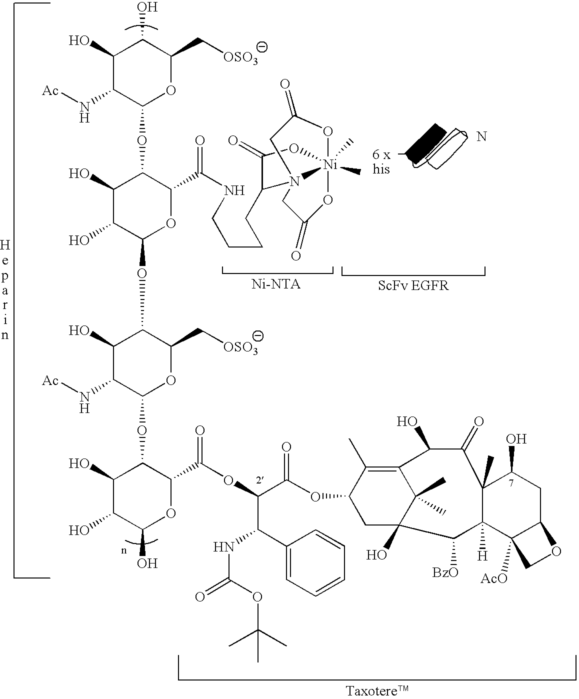

Peptides and proteins, including antibodies, also can be covalently coupled to a nanocarrier. For example, a native chemical ligation technique, such as described by Kent et al. in Chemical protein synthesis by solid phase ligation of unprotected peptide segments. J. Am. Chem. Soc. 121, 8720-27 (1999), can be used, as can the Staudinger ligation protocols discussed above.

Additional techniques for coupling materials, including those having the functional groups listed in Table 1, are taught by R. F. Taylor, (1991), “Protein immobilisation. Fundamental and applications”, Marcel Dekker, N.Y.; S. S. Wong, (1992), “Chemistry of Protein Conjugation and Crosslinking”, CRC Press, Boca Raton; and G. T. Hermanson et al., (1993), “Immobilized Affinity Ligand Techniques”, Academic Press, N.Y. Each of these publications is incorporated herein by reference.

Typically, the components of the nanocarrier compounds are directly bonded together without use of a spacer or linker component. For example, a therapeutic agent, an imaging agent and/or a targeting agent is directly bonded to the nanocarrier. However, in certain embodiments coupling of the conjugates disclosed herein include a linker covalently or non-covalently linking at least one of a therapeutic agent to the nanocarrier, the nanocarrier to a targeting agent, and a therapeutic agent to a targeting agent. In particular examples, the linker forms a covalent linkage between these agents, and thus comprises two or more reactive moieties, e.g. as described above, connected by a spacer element. The presence of such a spacer permits bifunctional linkers to react with specific functional groups within a molecule or between two different molecules, resulting in a bond between these two components and introducing extrinsic linker-derived material into the conjugate. The reactive moieties in a linking agent may be the same (homobifunctional agents) or different (heterobifunctional agents or, where several dissimilar reactive moieties are present, heteromultifunctional agents), providing a diversity of potential reagents that may bring about covalent bonding between any chemical species, either intramolecularly or intermolecularly.

In certain embodiments the disclosed conjugate compounds are used as prodrugs that deliver a therapeutic agent to a target. Thus it may be desirable to introduce labile linkages, e.g. containing spacer elements that are biodegradable or chemically sensitive or which incorporate enzymatic cleavage sites. In general, the presently disclosed conjugates may contain cleavable groups such as vicinal glycol, azo, sulfone, ester, thioester or disulphide groups linking two or more of the targeting, nanocarrier, and therapeutic or imaging components. In one embodiment, such groups are readily biodegraded in the presence of esterases in vivo, but are stable in the absence of such enzymes. Thus, the linkers can include such labile groups.

Linkers can include, for example, ethylene glycol, propylene glycol, ethanolamine, ethylenediamine, oligomers and derivatives thereof. Other representative spacer elements include oligosaccharides and polysaccharides, such as polygalacturonic acid, glycosaminoglycans, heparinoids, cellulose, alginates, chitosans carrageenans, dextran, aminodextran; peptides, polyamino acids and esters thereof, as in homo- and co-polymers of lysine, glutamic acid and aspartic acid; and oligonucleotides. In certain embodiments such linkers may contain enzyme cleavage sites.

Spacer elements may typically consist of aliphatic chains that optionally are interrupted by one or more heteroatoms and effectively separate the reactive moieties of the linker by distances of between about 0.5 and 300 nanometers. In one embodiment the spacer elements include polyethylene glycol derivatives, such as oligoethylene glycol and polyethylene glycol. Such polymeric structures, hereinafter referred to as PEGs, are simple, neutral polyethers which have been given much attention in biotechnical and biomedical applications (Milton Harris, J. (ed) “Poly(ethylene glycol) chemistry, biotechnical and biomedical applications” Plenum Press, New York, 1992). PEGs are soluble in most solvents, including water, and are highly hydrated in aqueous environments, with two or three water molecules bound to each ethylene glycol segment; this hydration phenomenon has the effect of preventing adsorption either of other polymers or of proteins onto PEG-modified surfaces. Furthermore, PEGs may readily be modified and bound to other molecules with only little effect on their chemistry. Their advantageous solubility and biological properties are apparent from the many possible uses of PEGs and copolymers thereof, including block copolymers such as PEG-polyurethanes and PEG-polypropylenes. Appropriate molecular weights for PEG spacers used in the presently disclosed conjugates typically are from about 120 Daltons to about 20 kilodaltons.

V. Compositions and Methods

Another aspect of the disclosure includes pharmaceutical compositions prepared for administration to a subject and which include a therapeutically effective amount of one or more of the currently disclosed compounds. Disclosed also are methods for administering the disclosed compounds and compositions. The therapeutically effective amount of a disclosed compound will depend on the route of administration, the type of mammal that is the subject and the physical characteristics of the subject being treated. Specific factors that can be taken into account include disease severity and stage, weight, diet and concurrent medications. The relationship of these factors to determining a therapeutically effective amount of the disclosed compounds is understood by those of ordinary skill in the art.

Methods are disclosed herein for treating conditions characterized by abnormal or pathological proliferative activity. Such conditions that can be treated according to the disclosed method include those characterized by abnormal cell growth and/or differentiation, such as cancers and other neoplastic conditions. Typical examples of proliferative disorders that can be treated using the disclosed compounds and compositions include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia, myelodysplasia, sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, bladder carcinoma, and CNS tumors (such as a glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma), and the like.

Methods also are disclosed herein for treating non-cancerous conditions. For example, methods are disclosed herein for improving vascular function in a subject. The methods include administering to the subject a therapeutically effective amount of a compound disclosed herein to improve vascular function. In one embodiment, the subject has atherosclerosis.

The therapeutically effective amount of the compound or compounds administered can vary depending upon the desired effects and the factors noted above. Typically, dosages will be between about 0.01 mg/kg and 250 mg/kg of the subject's body weight, and more typically between about 0.05 mg/kg and 100 mg/kg, such as from about 0.2 to about 80 mg/kg or from about 5 to about 40 mg/kg of the subject's body weight. Thus, unit dosage forms can be formulated based upon the suitable ranges recited above and a subject's body weight. In one embodiment, a therapeutically effective amount is effective to treat a condition associated with cardiovascular dysfunction (for example atherosclerosis). In one such embodiment, a therapeutically effective amount is an amount sufficient to increase blood flow.

The compounds disclosed herein may be administered orally, topically, transdermally, parenterally, via inhalation or spray and may be administered in dosage unit formulations containing conventional non-toxic pharmaceutically acceptable carriers, adjuvants and vehicles.

Typically, oral administration or administration via injection is preferred. The inhibitors may be provided in a single dosage or chronically, dependent upon the particular disease, condition of patient, toxicity of compound and other factors as will be recognized by a person of ordinary skill in the art.

The therapeutically effective amount of the compound or compounds administered can vary depending upon the desired effects and the factors noted above.

Pharmaceutical compositions for administration to a subject can include carriers, thickeners, diluents, buffers, preservatives, surface active agents and the like in addition to the molecule of choice. Pharmaceutical compositions can also include one or more additional active ingredients such as antimicrobial agents, anti-inflammatory agents, anesthetics, and the like. Pharmaceutical formulations can include additional components, such as carriers. The pharmaceutically acceptable carriers useful for these formulations are conventional. Remington's Pharmaceutical Sciences, by E. W. Martin, Mack Publishing Co., Easton, Pa., 19th Edition (1995), describes compositions and formulations suitable for pharmaceutical delivery of the compounds herein disclosed.

In general, the nature of the carrier will depend on the particular mode of administration being employed. For instance, parenteral formulations usually contain injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

It is specifically contemplated in some embodiments that inhibitor delivery is via an injected and/or implanted drug depot, for instance comprising multi-vesicular liposomes such as in DepoFoam (SkyePharma, Inc, San Diego, Calif.) (see, for instance, Chamberlain et al. Arch. Neuro. 1993, 50, 261-264; Katri et al. J. Pharm. Sci. 1998, 87, 1341-1346; Ye et al., J. Control Release 2000, 64, 155-166; and Howell, Cancer J. 2001, 7, 219-227).

A method is provided herein for the in vivo or in vitro detection of particular tissues or cells. An in vivo detection method can localize any target tissue or cell, such as atherosclerotic lesions, neo-vascularized and inflamed tissue areas, or a tumor, selected by choice of an appropriate targeting agent. In one embodiment, a subject having or suspected of having cancer is treated with a conjugate compound including an imaging agent. After a sufficient amount of time for the conjugate to localize to the tumor or cell in the subject, the tumor or cell can be detected. In one specific, non-limiting example detection of a cancer cell is accomplished using a technetium-99m labeled conjugate. Other specific, non-limiting examples of detection include fluorescence imaging.

In one specific embodiment, the detection step is performed prior to surgery. In another embodiment, the detection step is performed during surgery, for example to detect the location of the tumor prior to removing it, as in radioimmunoguided surgery.

In yet another embodiment, the detection step is performed after surgery to ensure the complete removal of the tumor, or to detect a recurrence of the tumor. In one specific, non-limiting example, a radiolabeled immune complex is detected using a hand-held gamma detection probe.

The in vitro detection method can be used to screen any biological sample containing any tumor or cell that expresses a targeted group as discussed below. Such samples include, but are not limited to, tissue from biopsies, autopsies, and pathology specimens. Biological samples also include sections of tissues, such as frozen sections taken for histological purposes. Biological samples further include body fluids, such as blood, serum, saliva, or urine. A biological sample is typically obtained from a mammal, such as a human subject. In one embodiment the subject has a condition comprising breast cancer, bladder cancer, bone cancer, cervical cancer, colon cancer, central nervous system cancer, esophageal cancer, gall bladder cancer, gastrointestinal cancer, head and neck cancer, laryngeal cancer, leukemia, lung cancer, melanoma, ovarian cancer, prostate cancer or renal cancer.

### EXAMPLES

The foregoing disclosure is further explained by the following non-limiting examples.

### General Methods

All reagents were of analytical grade. Paclitaxel was purchased from Hande Tech (Houston, Tex.). Heparin sodium of different molecular weights (15,000 and 5,000 dalton) was purchased from Celsus Laboratories, Inc. (Cincinnati, Ohio). Dicyclohexylcarbodiimide (DCC), FA, ethylene diamine, N-hydroxysuccinimide (NHS), and pyridine was obtained from Sigma (Milwaukee, Wis.). Anhydrous diethyl ether, acetonitrile, formamide and dimethylsulfoxide (DMSO) were purchased from Merck. (Darmstadt, Germany). Penicillin-streptomycin, fetal bovine serum (FBS), 0.25% (w/v) trypsin-0.03% (w/v) EDTA solution, and EMGM medium were purchased from American Type Culture Collection (Rockville, Md.). RPMI-1640 medium (without FA) was obtained from Invitrogen (Carlsbad, Calif.). Sephadex A-25 was purchased from Pharmacia Biotech AB (Uppsala, Sweden). Paclitaxel-Oregon Green 488 was purchased from Molecular Probes (Eugene, Oreg.).

### Example 1

### Synthesis of Conjugate Compounds

This example describes the synthesis of a Paclitaxel-Heparin-FA conjugate as illustrated in Scheme 1, below. Heparin (1 mmol) was activated using DCC (20 mmol) and NHS (22 mmol) in formamide at 4° C. overnight. Dicyclohexylurea (DCU) was removed by filtration and then heparin-NHS was obtained by recrystallization. The activated heparin-NHS (1 mmol) and aminated FA (20 mmol) were reacted at room temperature for 1 day. FA was aminated using conjugation with ethylene diamine. The unreacted aminated FA was removed by dialysis (molecular weight cut-off 2000). The final yellowish product was obtained by freeze-drying. The yield of conjugation was 95% (w/w). After dissolving the heparin-FA conjugate (1 mmol) in formamide, paclitaxel (30 mmol) and DCC (30 mmol) in DMSO were added. The mixture was reacted overnight at room temperature. After the reaction, recystallization and filtration were done to remove the unreacted DCC. For further purification, this product was precipitated by adding an excess volume of acetonitrile. After filtration, the yellow powder was dried in vacuum. The reaction was confirmed by TLC analysis (silica plate; eluent, 2-propanol/chloroform 10:90 vol:vol) and ¹H-NMR (400 MHz, in D₂O). To aminate FA, FA (1 mmol) dissolved in 30 ml DMSO was reacted with DCC (1 mmol) and NHS (2 mmol) at 60° C. for 8 h. The resulting FA-NHS was mixed with ethylene diamine (10 mmol) and 200 μl pyridine and allowed to react at room temperature overnight. The reaction was confirmed by TLC analysis (silica gel plate, 2-propanol/chloroform, 70:30 v/v %). The crude product was precipitated by addition of excess acetonitrile, filtered and washed three times with diethyl ether before drying under vacuum. For further purification, this product was dissolved in 2 NHCl and precipitated by adding an excess volume of acetonitrile. After filtration, the fine dark yellow powder was dried in vacuum. The unreacted FA and diaminated FA (FA-(NH₂)₂) were separated by ion-exchange chromatography. The column (10×200 mm) was packed by swollen DEAE Sephadex A-25 in 0.5 M potassium tetraborate solution. After dissolving in 30 ml of deionized water, the product (0.03 mg/ml) was loaded into the column, the linear ionic gradient of ammonium bicarbonate solution (10 to 30 mM) was applied. The FA-NH₂ solution was fractionally collected after continuous TLC analysis as mentioned above. The aminated FA solution was evaporated.

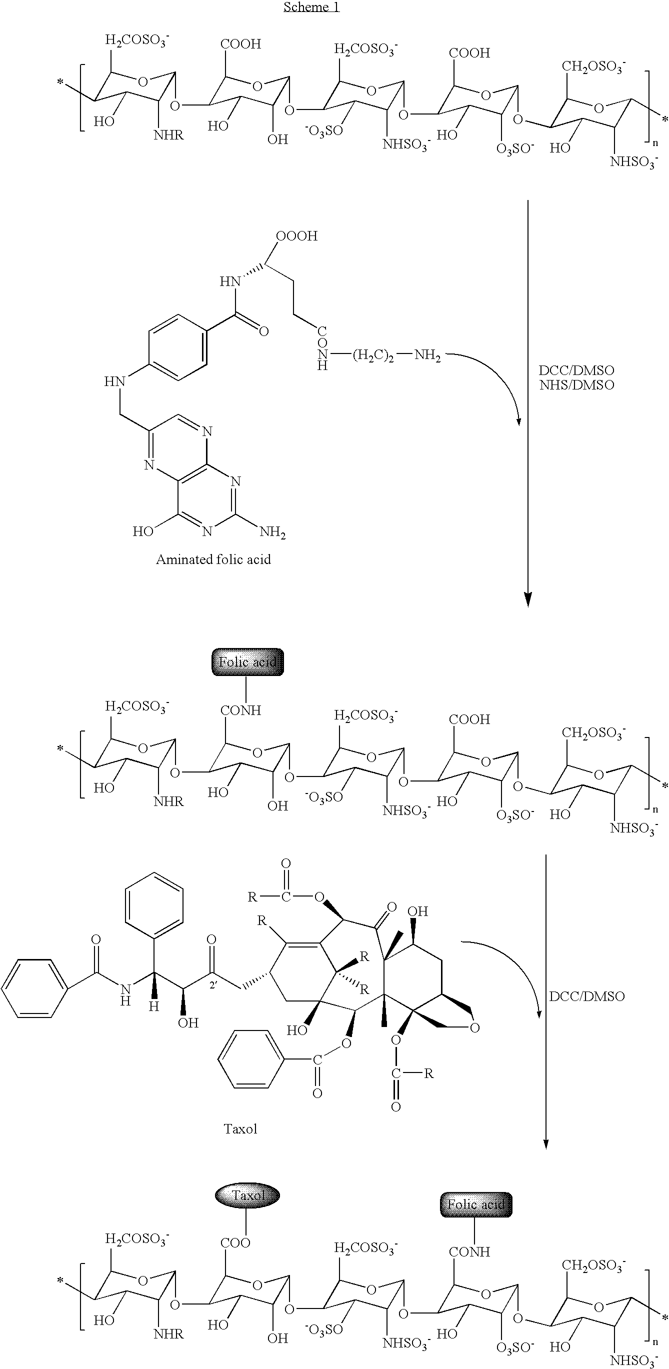

Observed signals for ¹H-NMR of paclitaxel (CDCl₃) were: ε1.11 [s, ¹⁷CH₃], 1.20 [s, ¹⁶CH₃], 1.69 [s, ¹⁹CH₃], 1.97 [s, ¹⁸CH₃], 2.2 [m, OAc], 2.4 [m, OAc], 3.78 [d, ³CH], 4.17 [d, ²⁰CH₂], 4.27 ppm [H-α, PG], 4.3 [d, ²⁰CH₂], 4.39 [dd, ⁷CH], 4.96 [d, ⁵CH], 4.78 and 5.63 [d, ^{2′}CH], 5.67 [d, ²CH], 5.98 [dd, ^{3′}CH], 6.22 [t, ¹³CH], 6.27 [s, ⁵CH], 7.09 [d, NH], 7.25 [s, 3′-Ph], 7.4 [m, 3′-NBz], 7.5 [m, 2-OBz], 7.73 [d, 3′-NBz], 8.1 [d, 2-OBz]. Values for ¹H-NMR of heparin(D)₂O) were: 85.38 [H1 of glucosamine residue(A)], 85.04 [H1 of iduronic acid residue(I)], ε4.84 [I-5], δ4.36-4.23 [A-6], δ4.12-4.40 [I-3], δ4.08-[I-4], δ4.02-[A-5], δ3.78-[I-2], δ3.71-[A-4], δ3.65-3.69-[A-3], δ3.24-[A-2]. Values for ¹H-NMR of aminated FA (DMSO) were: δ1.1 [s, C7-H of FA, 1H], δ4.28-4.16 [m, α-CH₂ of Glutamate of FA, 1H], δ6.64-[d, 3′,5′-H of FA, 2H], δ7.64-[d, 2′,6′-H of FA, 2H]. δ8.1-8.17 [H of CONH]. Values of 1H-NMR of Paclitaxel-Heparin-FA (D²O) were δ1.11-2.4 [CH₃ or OAc of paclitaxel], δ3.24-5.38-[A or I of heparin], δ7.5-7.64 [2-OBz and NBz of paclitaxel], 7.64-[H of FA, 2H], δ8.1-8.45 [CONH between heparin and FA] and δ5.6 [COO between heparin and paclitaxel]

### Example 2

### Characterization of Conjugate Compounds

This example describes the characterization of the Paclitaxel-Heparin-FA produced according to example 1. UV-vis absorption spectra were recorded on a Shimadzu UV-2401PC scanning spectrophotometer operating at a slit width of 1.0 nm. The content of paclitaxel conjugated to heparin-FA was estimated by LUV measurements based on a standard curve generated with known concentrations of paclitaxel in methanol (λ=228 nm). The IR spectra of Paclitaxel-Heparin-FA were acquired on a Fourier transform infrared spectroscopy (FT-IR) using a Perkin Elmer system 2000 spectrometer and the samples were analyzed as KBr pellets.

Synthesis of heparin-FA was confirmed by the presence of signals at δ6.75-8.77 ppm in the ¹H-NMR spectrum of Heparin-FA and by an absorbance at λ=280 nm in the UV spectrum of the heparin-FA. Coupling of the paclitaxel to heparin-FA was achieved via a DCC mediated reaction of hydroxyl groups of paclitaxel and the carboxyl groups of heparin. The formation of the linkage was confirmed by the presence of signals at δ5.6 ppm in the ¹H-NMR spectrum. Both C-2′ proton and C-7 proton were exactly overlapped with the peaks of heparin. The specific site of esterification between paclitaxel and heparin may be both the C-2′ and C-7 positions. From the FT-IR spectrum, the binding of heparin-FA and Paclitaxel was confirmed by the presence of ester groups around 1732 cm⁻¹ (C═O ester bond) in Paclitaxel-Heparin-FA. The UV spectrum of Paclitaxel-Heparin-FA conjugate in water displayed a slight shift (λmax=210 nm) compared with that of paclitaxel in methanol (λmax=228 nm). Paclitaxel-Heparin-FA in water produced a clear solution at a concentration of 50 mg/ml. The amount of paclitaxel conjugated to Heparin-FA estimated by TV spectra based upon the standard curve was 15.4% by weight. There are 50 COOH groups in heparin (molecular weight: 15000 dalton) and all were completely functionalized by paclitaxel and FA.

The average particle size, size distribution and morphology were examined using a particle size analyzer (Brookhaven Instruments Co., model 90Plus, Holtsville, N.Y.) and a TEM (Hitachi, model H-600, Japan) at a voltage of 80 kV. The aqueous dispersion of the particles was drop-cast onto a carbon coated copper grid and grid was air dried at room temperature before loading into the microscope. Samples for the size measurements were prepared by dispersing small amounts of the Paclitaxel-Heparin-FA powder in water and treating in an ultrasonic bath for 1 min. The measurement conditions were employed a wavelength of 656 nm, viscosity of 0.89 cp, and refractive index of 1.33.

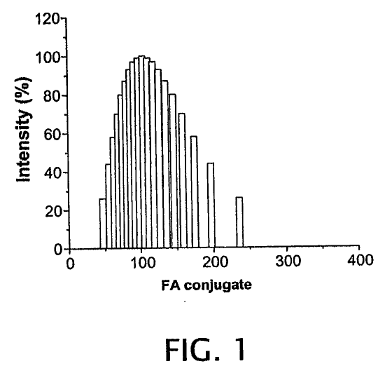

With reference to FIG. 1, the mean diameter of the nanoparticles as measured by light scattering was 118.3 nm with a standard deviation of 2.4 nm. TEM micrograph of the Paclitaxel-Heparin-FA conjugate was taken to determine the shape and uniformity of the particles, and the TEM micrographs demonstrated that the particles have a uniform spherical shape (micrographs not shown). When low molecular weight heparin (molecular weight 5000) was modified with paclitaxel and FA, the size distribution of the particle in water was around 1200 nm in diameter (data not shown).

The bioactivities of heparin and Paclitaxel-Heparin-FA conjugate measured by factor Xa chromogenic assay were 179 and 68 IU/mg, respectively as illustrated in FIG. 2. The relative bioactivity of Paclitaxel-Heparin-FA was 38% of the unmodified heparin. Because of the presence of sulfonyl, carboxyl and hydroxyl groups in the active site of heparin, the activity of Paclitaxel-Heparin-FA conjugate was dramatically decreased. Because the anticoagulant activity of the Paclitaxel-Heparin-FA conjugates is low, more conjugates can reach the cancer cells because unspecific binding affinity decreases. Side effects such as bleeding and thrombocytopenia associated with excess dosage of heparin should not be problematic. Furthermore, binding sites for growth factor are still intact as 2-O-sulfate and 6-O-sulfate in heparin are available for binding with VEGF and FGF. See, Ashikari-Hada S.; Habuchi H.; Kariya Y.; Itoh N.; Reddi A. H.; Kimata K. J. Biol. Chem. 2004, 279, 12346 and Leamon C P.; Cooper S R.; Hardee G E. Bioconjugate Chem. 2003, 14, 738-747.

### Example 3

### Characterization of Tubulin Binding Activity

This example describes the tubulin polymerization assay used to evaluate the disclosed compounds. The tubulin assembly reaction was performed in G-PEM buffer (1 mM GTP, 80 mM PIPES, 1 mM EGTA, 0.5 mM magnesium chloride; pH 6.8) at a tubulin (Cytoskeleton Inc., Boulder, Colo.) concentration of 1 mg/ml (10 μM) in the presence of drugs (10 μM). The instrument was zeroed with this solution at 4° C. Paclitaxel or heparin-Paclitaxel conjugates were then quickly mixed into the tubulin solution to a final concentration of 10 μM and the absorbance was continually monitored over an 80 min period. These samples were placed in quartz cuvettes and incubated at 32° C. Tubulin polymerization was observed by measuring the absorbance of the solution (340 nm).

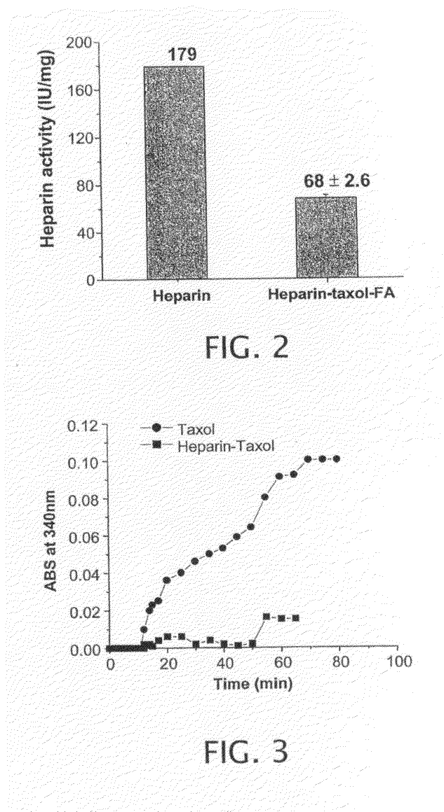

With reference to FIG. 3, the ability of paclitaxel and Paclitaxel-Heparin-FA conjugate to induce microtubule assembly in vitro was determined at 10 μM paclitaxel or heparin-paclitaxel conjugate. The addition of paclitaxel to a solution of tubulin in an assembly buffer caused a clear increase in absorbance due to the increase in light scattering that resulted from the polymerization of tubulin into microtubules. On the other hand, 10 μM paclitaxel equivalent of heparin-paclitaxel had no effect on polymerization as shown in FIG. 3. Thus, the ability of paclitaxel to induce microtubule assembly dramatically decreased due to acylation of the hydroxyl group with heparin. Nonetheless, the presently disclosed paclitaxel conjugates are designed to release paclitaxel as an active drug after being cleaved from Paclitaxel-Heparin-FA. Thus, active paclitaxel is released at the target.

### Example 4

### Evaluation of Cytotoxicity

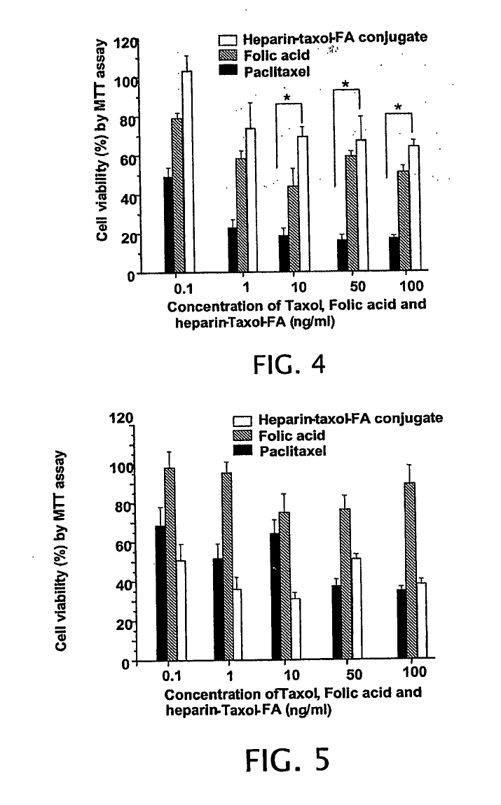

This example describes the cytotoxicity assay used to evaluate the compounds disclosed herein. Human nasopharyngeal epidermoid carcinoma, KB cells and human breast cells, MCF-10A, cell lines were purchased from American Type Culture Collection (ATCC, Manassas, Va.). These cell lines were selected because of the positive FR overexpression (KB) or the lack of a detectable FR expression (MCF-10A). The KB cell lines were cultured at 37° C. in a humidified atmosphere containing 5% CO₂ in FA deficient medium RPMI 1640 with 10% fetal calf serum. The MCF-10A cell lines were cultured at 37° C. in a humidified atmosphere containing 5% CO₂ in medium MEGM (Mammary epithelial growth medium, serum-free) supplemented with cholera toxin. The cells (4×10⁴ cells/ml) grown as a monolayer were harvested by 0.25% trypsin-0.03% EDTA solution. The cells (200 μl) in their respective media were seeded in a 96-well plate and preincubated for 24 h before the assay. MTT assay was performed on KB cells and MCF-10A cells by incubating at 37° C. for 2 days with five different concentrations (0.1, 1 10, 50 and 100 ng/ml) of the each compound in quadruplicate. The control was incubated at 37° C. for 2 days without adding a drug. This assay is based on the reduction of the yellow tetrazolium component (MTT) to an insoluble purple-colored formazan produced by the mitochondria of viable cells. After a 48-hr incubation, 10 ul of medium containing 20 ul of MTT solution was added to each well and the plate was incubated for an additional 4 h, followed by the addition of 100 ul of MTT solublization solution (10% Triton X-100 plus 0.1 N HCl in anhydrous isopropanol, Sigma, Milwaukee, Wis.) to each well. The solution was gently mixed to dissolve the MTT formazan crystals. The absorbance of each well was read with a microplate reader at a wavelength of 570 nm. The background absorbance of well plates at 690 nm was measured and subtracted from the 570 nm measurement. The results are expressed as % cell viability, obtained by dividing the optical density values (OD) of the treated groups (T) by the OD of the controls (C) ([T/C×100%, FIG. 4). The concentration of the agent causing 50% cytotoxicity (IC₅₀) and the 95% confidence intervals were calculated by nonlinear regression of log-transformed data. Statistical analysis was done using ANOVA. p<0.01 was accepted as statistically significant. Error bars represent standard error of mean (SEM).

FIGS. 4 and 5 record the cell viability of MCF-10A cells (normal breast cells) and KB cells (cancer cells), respectively after a 48 hour incubation with paclitaxel, folic acid and Paclitaxel-Heparin-FA. The data recorded in FIGS. 4 and 5 demonstrate that the disclosed compounds are cytotoxic to and selective for malignant cells over normal cells. The IC₅₀ values observed reveal that the Paclitaxel-Heparin-FA conjugate has increased cytotoxic potential than paclitaxel alone and greater selectivity against malignant cells over normal cells, which is consistent with an activity based on paclitaxel release. Overall, the results of the MTT assay indicate that Paclitaxel-Heparin-FA is not cytotoxic to normal cells but is highly toxic to cancer cells.

Paclitaxel-Heparin-FA with a negative response had an IC₅₀ of >0.1 ng/ml in cancer cells, indicating the nanoparticle can be taken up by the KB cells via folate receptor-mediated endocytosis. Paclitaxel-Heparin-FA nanoparticles are not cytotoxic to normal cells because the normal cells express a lower folate-receptor concentration and hence are not targeted by the paclitaxel conjugate. When normal cells were treated with free folate, viability of the cells was relatively low compared to cells treated with Paclitaxel-Heparin-FA. Thus the present strategy minimizes the toxicity of both the targeting agent and the therapeutic agent.

### Example 5

### Visualization of Cellular Uptake

This example describes the visualization of the cellular uptake and intracellular distribution of labelled Paclitaxel-Heparin-FA conjugates. Confocal laser scanning microscopy (Zeiss LSM510, Germany) was performed with KB cells grown on a Lab-Tek® II chamber slide (Nalge Nunc, Naperville, Ill.). Paclitaxel-Oregon Green® 488 conjugate (Molecular Probes, Eugene, Oreg.) was used to conjugate with heparin or heparin-FA as described above. The concentration of labelled Paclitaxel-Heparin-FA conjugates was 1 μg/ml in medium RPMI-1640. After 1 hr incubation, the medium containing the complexes was aspirated from the wells. The cells were then washed three times with PBS buffer (pH 7.4) and finally 200 μl of 4% formaldehyde in phosphate buffer saline was added. The samples were observed as quickly as possible. Initial scanning of the tissue sections was done under low power magnification (10× lens) with conventional fluorescence microscopy and the FITC filter. The FITC labeled areas were then scanned with the confocal microscope (excitation/emission wavelengths: 488 nm and 510 nm) at higher magnification (100× lens).

Significant amounts of labeled Paclitaxel-Heparin-FA were clearly observed to associate with KB cells. Large and numerous punctate fluorescent structures were found in all observed cells. On the other hand, a dramatic reduction in cell association was observed for FITC-heparin or FITC-heparin-paclitaxel conjugate lacking a conjugated FA moiety. These data demonstrate that Paclitaxel-Heparin-FA can efficiently target FR-positive cells. Treatment of KB cells with 1 μg/ml of Paclitaxel-Heparin-FA mixed with 10 μg/ml of free FA resulted in nearly complete displacement of Paclitaxel-Heparin-FA, demonstrating proving receptor specific binding of the conjugate.

### Example 6

### In vivo Evaluation of Conjugates

This example describes the inhibition of tumor growth in mice implanted with human tumor cells.

Human Tumor Xenograft: Six to seven-week-old Crl:NU/NU-nuBR female nude mice (21-25 g) were purchased from Charles River Laboratories Inc. (Wilmington, Mass.) and maintained under specific pathogen-free conditions. All experiments were approved by the Emory University's Institutional Animal Care and Use Committee in accordance with the NIH Guidelines. Cultured KB cells (ATCC, Rockville, Md.) were trypsinized, washed twice with serum-free RPMI1640, and suspended at 5×10⁷ cells/ml PBS. A quantity of 100 μl of suspended cells was subcutaneously injected into the back of the mice. On day 12-15 after tumor injection, the resulting tumors reached a volume of 80-100 mm³. According to body weight and tumor size, the animals were divided into five experimental groups of five mice each: group A, B, C, D and E were respectively treated by intravenous tail vein injections of with 100 μl of saline as control (Group A, n=4), paclitaxel (80 mg/kg, Group B, n=4), heparin (or PG)-paclitaxel-FA (40 and 80 mg/kg, Group C and D, n=4), and heparin (or PG)-paclitaxel (80 mg/kg) plus free FA (20 mg/kg, Group F, n=4).

Each drug was given at 7-day intervals for a total of 4 injections after tumor inoculation. Mice were sacrificed when they became moribund because of tumor burden or when they had weight loss of >25%. The experiment was terminated on day 25. Tumor growth was determined by measuring three orthogonal tumor diameters. Tumor volume was calculated as follows: Volume=π/6×length (mm)×width (mm)×height (mm). In order to evaluate drug toxicity in non-tumor bearing mice, two dosages (50 and 100 mg/kg) of heparin (or PG)-paclitaxel-FA or paclitaxel were injected intravenously every 7 days, respectively and body weights of mice were measured every 2-3 days.

Immunohistologic staining and histological analysis: Tumor tissues were embedded in O.C.T. compound (Miles, Elkhart, Ind., USA) and frozen in liquid nitrogen. Tissue sections of 5 μm were prepared on glass slides. Immunohistochemical staining to identify vasculature was performed using the endothelial specific marker CD31 (PECAM-1) on sections of the frozen-embedded disc using rat anti-mouse PECAM-1 (Santa Cruz Biotechnology Inc., Santa Cruz, Calif.) followed by FITC X (RRX)-labeled goat anti-rat IgG (Jackson ImmunoResearch Laboratories Inc, West Grove, Pa.). Nuclei Counter staining was performed with Hoechst 33258 (Sigma) to identify the areas of cell invasion. As a measure of angiogenesis green colored PECAM-1 positive areas were measured and normalized by the total cellular area determined by blue nuclear staining.

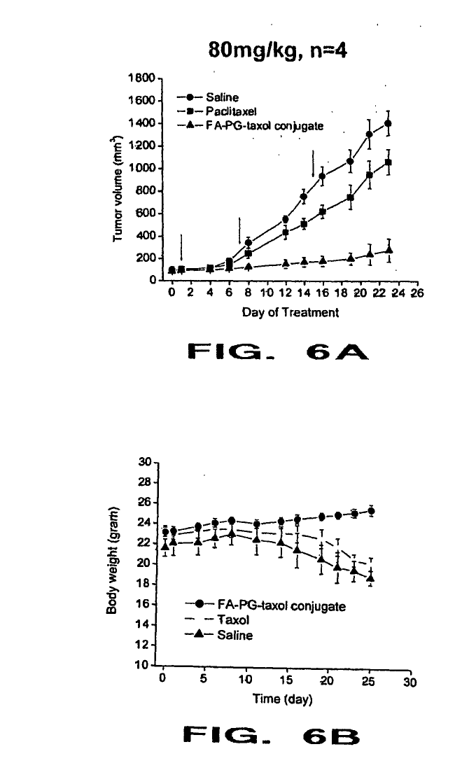

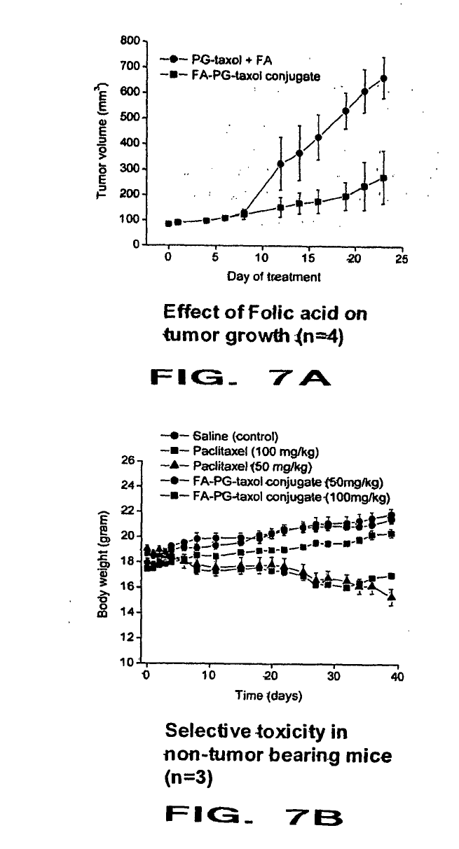

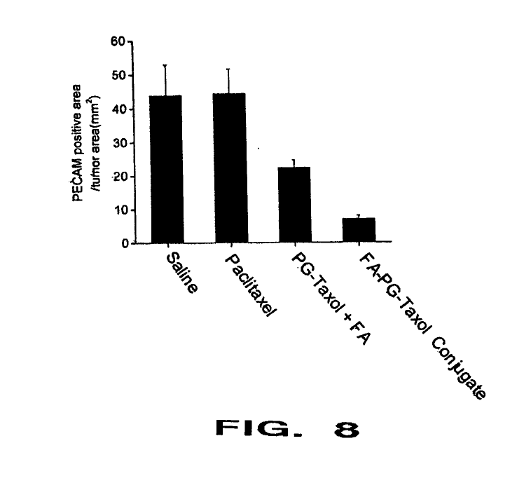

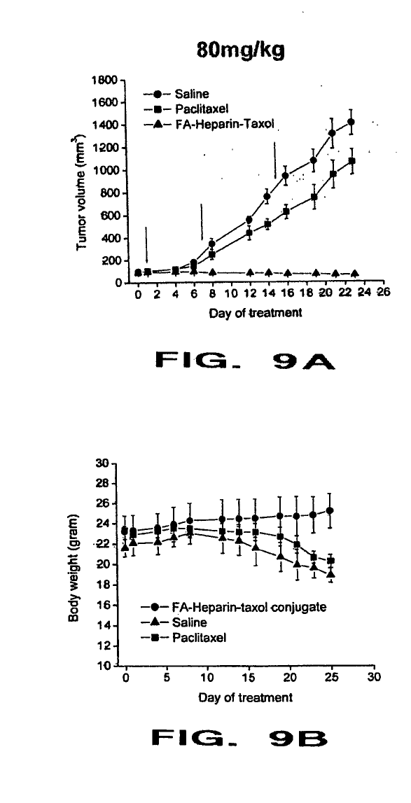

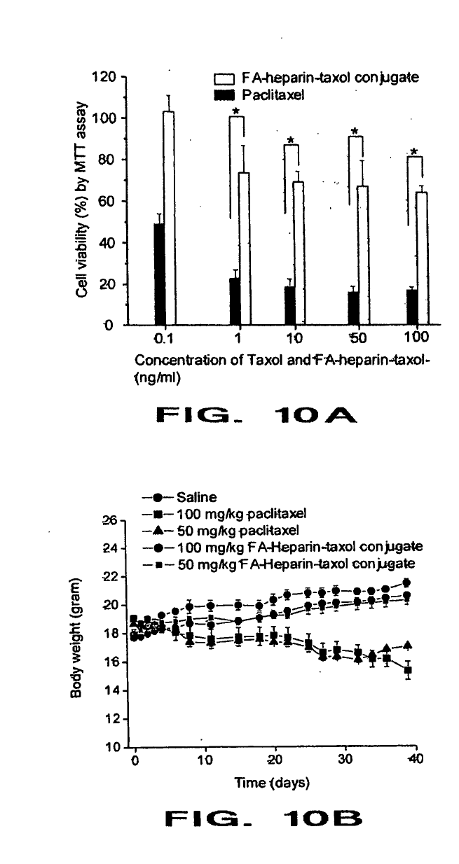

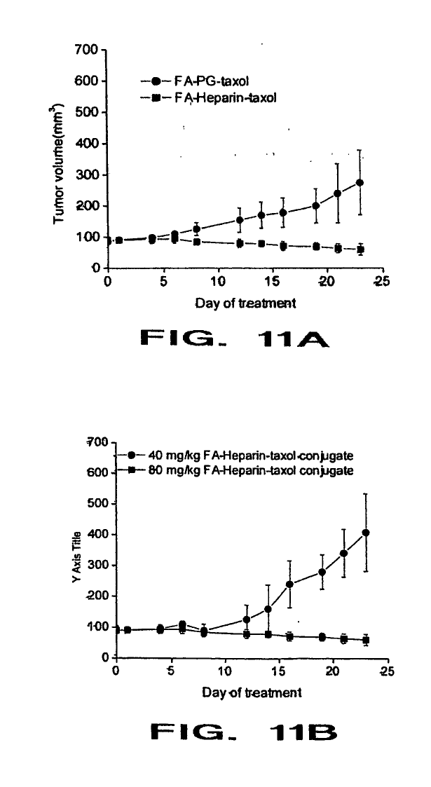

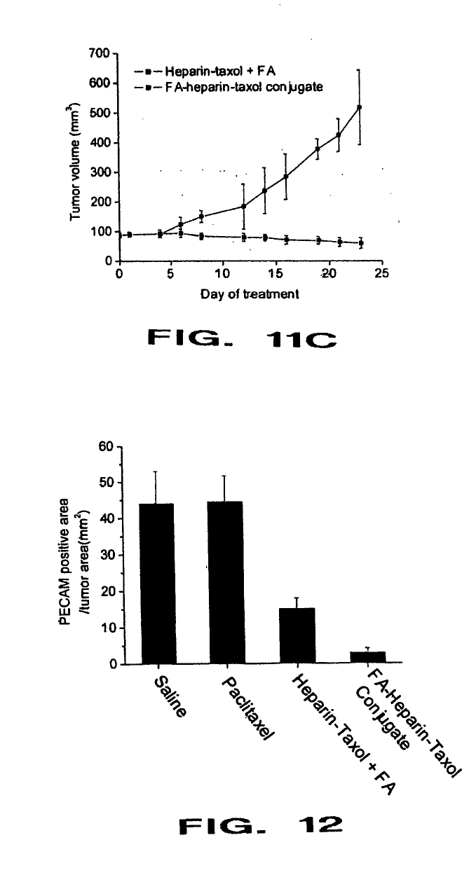

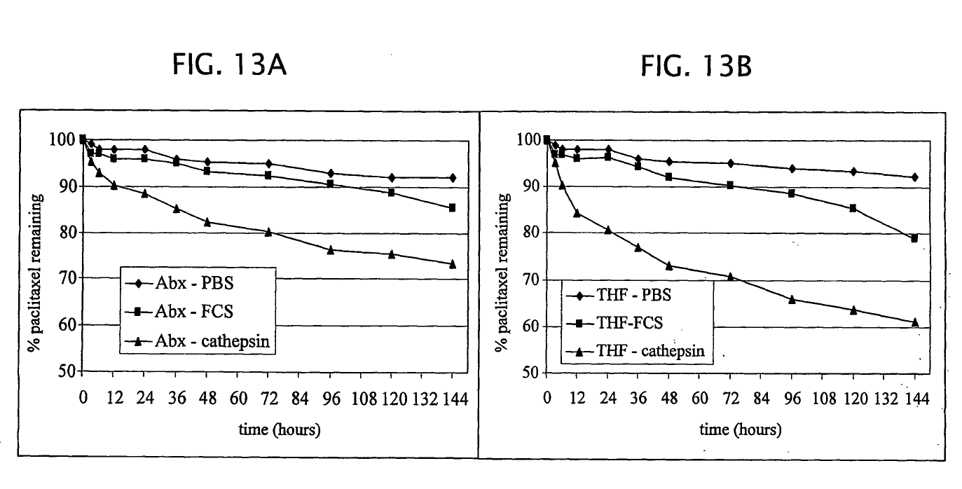

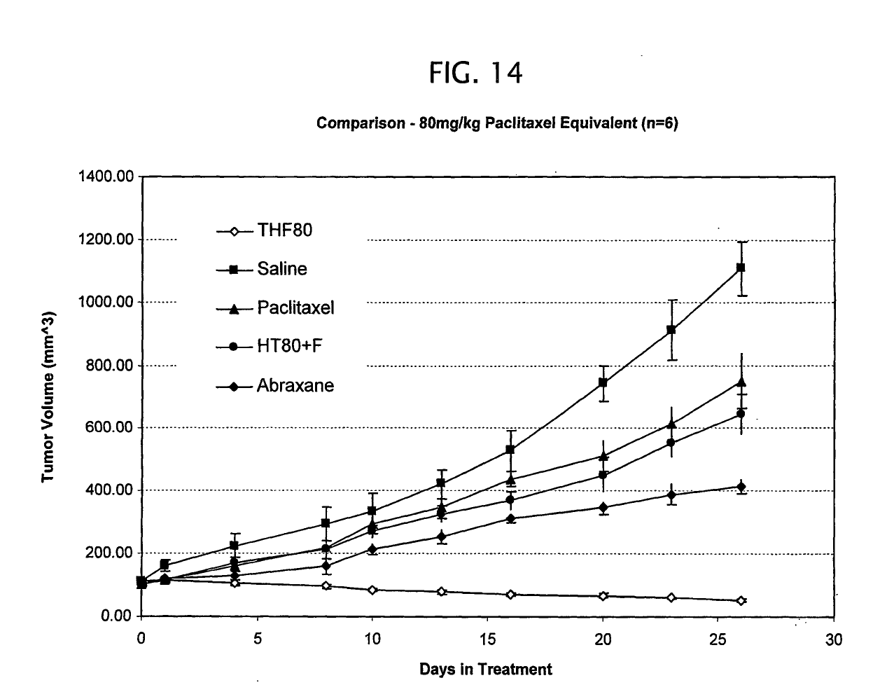

With reference to FIGS. 6A and 6B administration of paclitaxel-PG-FA to mice results in minimizes tumor growth, with maintenance of mouse body weight. These data demonstrate that paclitaxel-PG-FA is considerably more effective than paclitaxel alone. FIG. 7A demonstrates that the enhanced efficacy of paclitaxel-PG-FA results from folate receptor targeting. The data recorded in FIG. 7B demonstrate that paclitaxel-PG-FA exhibits selective toxicity in mice. FIG. 8 illustrates that paclitaxel-PG-FA reduces angiogenesis in tumors. FIGS. 9A and 9B demonstrate that administration of paclitaxel-heparin-FA to mice results in minimizes tumor growth, with maintenance of mouse body weight. FIGS. 10A and 10B demonstrate that paclitaxel-heparin-FA is relatively non-toxic to non-cancerous cells. FIG. 11A demonstrates that paclitaxel-heparin-FA is more effective preventing tumor growth than paclitaxel-PG-FA. FIG. 111B demonstrates that paclitaxel-heparin-FA is more effective at preventing tumor growth at a higher dosage. FIG. 11C demonstrates that the enhanced efficacy of paclitaxel-heparin-FA results from folate receptor targeting. FIG. 12 demonstrates that paclitaxel-heparin-FA reduces angiogenesis in tumors.

It will be apparent to those skilled in the art that various modifications and variations can be made in the present compounds, compositions and methods without departing from the scope or spirit of the disclosure. Other embodiments of the compounds, compositions and methods will be apparent to those skilled in the art from consideration of the specification and practice of the procedures disclosed herein. It is intended that the specification and examples be considered as exemplary only, with a true scope and spirit of the invention being indicated by the following claims.

We claim:

1. A conjugate comprising a nanocarrier, a therapeutic agent or imaging agent, and a targeting agent.

2. The conjugate according to claim 1, wherein the nanocarrier comprises a nanoparticle, an organic polymer, or both.

3. The conjugate according to claim 2, wherein the conjugate is a compound having the following formula:
A-X—Y
wherein A represents the chemotherapeutic agent or imaging agent;
X represents the nanoparticle, organic polymer or both, wherein the organic polymer has an average molecular weight of at least about 1,000 daltons; and
Y represents the targeting agent.

4. The conjugate according to claim 3, wherein the organic polymer comprises a polyamino acid, a polysaccharide, or combinations thereof.

5. The conjugate according to claim 3, wherein the organic polymer is a polyionic polymer.

6. The conjugate according to claim 3, wherein the organic polymer comprises heparin, hyaluronic acid, polyglutamic acid, polylysine, N-(2-hydroxypropyl)-methacrylamide copolymer, poly-2-hydroxyethylmethacrylate, poly(poly-2-hydroxyethylmethacrylate co-methacrylate), polystyrene, polyethylene glycol, polyoxamers, polyoxamines, poly(methyl methacrylate), poly(butyl 2-cyanoacrylate), dextran, carboxymethyl dextran, cellulose, carboxymethyl cellulose, chitosan, copolymers thereof or combinations thereof.

7. The conjugate according to claim 3, wherein X represents an organic polymer and the conjugate self assembles under physiological conditions to yield a self-assembled nanoparticle.

8. The conjugate according to claim 7, wherein the organic polymer comprises heparin.

9. The conjugate according to claim 7, wherein the self-assembled nanoparticle has a diameter of from about 1 nanometer to about 1500 nanometers.

10. The conjugate according to claim 7, wherein the self-assembled nanoparticle has a diameter of from about 1 nanometer to about 1200 nanometers.

11. The conjugate according to claim 7, wherein the self-assembled nanoparticle has a diameter of from about 10 to about 400 nanometers.

12. The conjugate according to claim 7, wherein the self-assembled nanoparticle has a diameter of from about 100 to about 250 nanometers.

13. The conjugate according to claim 3, wherein the nanoparticle is a polymeric nanoparticle.

14. The conjugate according to claim 13, wherein the nanoparticle comprises heparin, polyglutamic acid, polylysine, poly-2-hydroxyethylmethacrylate, N-(2-hydroxypropyl)-methacrylamide copolymer, poly(poly-2-hydroxyethylmethacrylate co-methacrylate), polystyrene, polyethylene glycol, polyoxamers, polyoxamines, polyphosphazene, poly(methyl methacrylate), poly(butyl 2-cyanoacrylate), copolymers thereof and combinations thereof.

15. The conjugate according to claim 14, wherein the nanoparticle comprises heparin.

16. The conjugate according to claim 3, wherein the organic polymer has a molecular weight of from about 1,000 to about 150,000 daltons.

17. The conjugate according to claim 3, wherein the organic polymer has a molecular weight of from about 5,000 to about 100,000 daltons.

18. The conjugate according to claim 3, wherein the organic polymer has a molecular weight of from about 10,000 to about 50,000 daltons.

19. The conjugate according to claim 3, wherein the nanoparticle comprises a metal cluster.

20. The conjugate according to claim 19, wherein the metal cluster comprises silver, gold, platinum, palladium, cobalt and/or iron.

21. The conjugate according to claim 3, wherein the nanoparticle has a diameter of from about 1 nanometer to about 1500 nanometers.

22. The conjugate according to claim 3, wherein the nanoparticle has a diameter of from about 1 nanometer to about 1200 nanometers.

23. The conjugate according to claim 3, wherein the nanoparticle has a diameter of from about 10 to about 400 nanometers.

24. The conjugate according to claim 3, wherein the nanoparticle has a diameter of from about 100 to about 250 nanometers.

25. The conjugate according to claim 3, further comprising plural therapeutic agents.

26. The conjugate according to claim 25, wherein the plural therapeutic agents are the same.

27. The conjugate according to claim 25, wherein at least two therapeutic agents are different.

28. The conjugate according to claim 3, wherein the therapeutic agent is selected from the group consisting of microtubule binding agents, DNA intercalators or cross-linkers, DNA synthesis inhibitors, DNA and/or RNA transcription inhibitors, enzyme inhibitors, gene regulators, anti-atherosclerosis agents and/or angiogenesis inhibitors.

29. The conjugate according to claim 3, wherein the therapeutic agent is selected from the group consisting of paclitaxel, docetaxel, daunorubicin, cisplatin, carboplatin, oxaliplatin, colchicine, dolastatin 15, nocodazole podophyllotoxin, rhizoxin, vinblastine, vindesine, vinorelbine (navelbine), the epothilones, the mitomycins, bleomycin chlorambucil, cannustine, melphalan, mitoxantrone 5-fluoro-5′-deoxyuridine, camptothecin, topotecan, irinotecanetoposide, tenoposide, geldanamycin, methotrexate, adriamycin, actinomycin D, mifepristone, raloxifene, 5-azacytidine, 5-aza-2′-deoxycytidine, zebularine, tamoxifen, 4-hydroxytamoxifen apigenin, rapamycin, angiostatin K1-3, staurosporine, genistein, fumagillin, endostatin, thalidomide, analogs thereof and combinations thereof.

30. The conjugate according to claim 3, wherein the targeting agent is selected from the group consisting of folate, biotin, vitamin B₁₂, and derivatives thereof.

31. The conjugate according to claim 3, wherein the targeting agent comprises a growth factor, cytokine, peptide, protein, antibody or an antibody fragment.

32. The conjugate according to claim 3, wherein the compound has a formula
Aₘ-X-Yₙ
A, X and Y are as described above; and
n and m independently are integers from 2 to about 500.

33. The conjugate according to claim 3, wherein the compound has a formula
Aₘ-X-Yₙ
A, X and Y are as described above;
m is from 2 to about 500; and
n is 1.

34. The conjugate according to claim 33, comprising a chemotherapeutic agent and an imaging agent.

35. The conjugate according to claim 3, wherein the compound has a formula
Aₘ-X-Yₙ
A, X and Y are as described above;
m is 1; and
n is from 2 to about 500.

36. The conjugate according to claim 3, further comprising a linker linking at least one of A to X and X to Y.

37. The conjugate according to claim 36, wherein the linker covalently links at least one of A to X and X to Y.

38. The conjugate according to claim 36, wherein the linker comprises a hydrocarbon chain, ethylene glycol, polyethylene glycol, polysaccharides, polypropylene oxide, hydroxyethyl amine, polyhydroxyethylamine or combinations thereof.

39. The conjugate according to claim 28, further comprising an imaging agent.

40. The conjugate according to claim 3, comprising an imaging agent.

41. The conjugate according to claim 40, wherein the imaging agent comprises a magnetic resonance contrast reagent, an optical imaging agent, a radioisotope or combinations thereof.

42. A pharmaceutical composition comprising a pharmaceutically acceptable carrier and a therapeutically effective amount of a conjugate according claim 3.

43. The composition according to claim 42, wherein the organic polymer comprises a polyamino acid, a polysaccharide, or combinations thereof.

44. The composition according to claim 42, wherein the organic polymer comprises heparin, hyaluronic acid, polyglutamic acid, polylysine, N-(2-hydroxypropyl)-methacrylamide copolymer, poly-2-hydroxyethylmethacrylate, poly(poly-2-hydroxyethylmethacrylate co-methacrylate), polystyrene, polyethylene glycol, polyoxamers, polyoxamines, poly(methyl methacrylate), poly(butyl 2-cyanoacrylate), dextran, carboxymethyl dextran, cellulose, carboxymethyl cellulose, chitosan, copolymers thereof or combinations thereof.

45. The composition according to claim 42, wherein X represents an organic polymer and the compound self assembles under physiological conditions to yield a self-assembled nanoparticle.

46. The composition according to claim 42, wherein the conjugate comprises an imaging agent.

47. A method for treating a subject having a hyperproliferative disorder, comprising administering to the subject a conjugate according to claim 2, thereby treating the subject.

48. The method according to claim 47, wherein the subject has a condition comprising breast cancer, bladder cancer, bone cancer, cervical cancer, colon cancer, central nervous system cancer, esophageal cancer, gall bladder cancer, gastrointestinal cancer, head and neck cancer, laryngeal cancer, leukemia, lung cancer, melanoma, ovarian cancer, prostate cancer or renal cancer.

49. The method according to claim 47, wherein the subject has breast cancer.

50. A method for delivering a therapeutic agent to a cell, comprising contacting the cell with a conjugate according to claim 1, thereby delivering the therapeutic agent.

51. The method according to claim 50, wherein the therapeutic agent is conjugated to the nanocarrier via a physiologically labile bond.

52. The method according to claim 51, wherein the bond is an ester bond.

53. The method according to claim 51, wherein delivering the chemotherapeutic agent comprises receptor-mediated endocytosis.

54. A composition comprising a plurality of self-assembled nanoparticles comprising conjugates according to claim 3.

55. The composition according to claim 54, further comprising a solvent.

56. The composition according to claim 54, further comprising a pharmaceutically acceptable buffer solution.

57. The composition according to claim 54, wherein the self-assembled nanoparticles have a diameter of from about 1 nanometer to about 1500 nanometers.

58. The composition according to claim 54, wherein the self-assembled nanoparticles have a diameter of from about 1 nanometer to about 1200 nanometers.

59. The composition according to claim 54, wherein the self-assembled nanoparticles have a diameter of from about 10 to about 250 nanometers.

60. The composition according to claim 54, wherein the self-assembled nanoparticles have a diameter of from about 100 to about 400 nanometers.

61. A compound comprising heparin, paclitaxel and folic acid.

62. The compound according to claim 61, wherein the compound comprises plural paclitaxel moieties and plural folic acid moieties.

63. The conjugate according to claim 1, wherein the conjugate has one of the following formulas:
A-X—Y
X-A-Y
or
X—Y-A
wherein A represents the chemotherapeutic agent or imaging agent;
X represents the nanoparticle, organic polymer or both, wherein the organic polymer has an average molecular weight of at least about 1,000 daltons; and
Y represents the targeting agent.

64. The conjugate according to claim 63, wherein the conjugate has a formula
Aₘ-X-Yₙ
X-Aₘ-Yₙ
or
X—Yₙ-Aₘ
wherein A, X and Y are as described above and n and m independently are integers from 2 to about 500.

65. The conjugate according to claim 64, further comprising a linker linking at least one of A to X, X to Y, and A to Y.
